(19) United States
(12) Patent Application Publication
(10) Pub. No.: US 2008/0305079 A1
(43) Pub. Date: Dec. 11, 2008

(54) Myeloid Suppressor Cells, Methods For Preparing Them, and Methods For Using Them For Treating Autoimmunity

(75) Inventor: Shu-Hsia Chen, New York, NY (US)

Correspondence Address: DARBY & DARBY P.C., P.O. BOX 770, Church Street Station, New York, NY, 10008-0770, US

(73) Assignee: Mount Sinai School of Medicine of New York University, New York, NY (US)

(21) Appl. No.: 12/159,929

(22) PCT Filed: Jan. 8, 2007
(86) PCT No.: PCT/US07/60210

§ 371 Date: Aug. 11, 2008

Related U.S. Application Data
(60) Provisional application No. 60/756,943, filed on Jan. 6, 2006.

(51) Int. Cl. A61K 38/20; A61K 35/14; A61K 38/21; A61P 37/00; C12N 5/02

(52) U.S. Cl. 424/85.2; 424/93.71; 435/378; 424/85.4

(57) ABSTRACT

The present invention relates to novel myeloid suppressor cells (MSCs) and to methods of isolating these MSCs are also included. The MSCs of the present invention can be used to treat or prevent autoimmune diseases or alloimmune responses. The MSCs of the present invention may also be used to reduce a T cell response, induce T regulatory cells, and produce T cell tolerance.

### CROSS-REFERENCE TO RELATED APPLICATION

Priority is claimed to U.S. Provisional Patent Application Ser. No. 60/756,943, filed on Jan. 6, 2006. The content of this priority application is incorporated into the present disclosure by reference and in its entirety.

### STATEMENT REGARDING FEDERALLY SPONSORED RESEARCH

The research leading to this invention was supported, in part, by the National Cancer Institute, Grant No. CA 70337, and the National Institutes of Health, Grant Nos. CA109322 and DK073603. Accordingly, the United States government may have certain rights to this invention.

### BACKGROUND

The key to a healthy immune system is its ability to distinguish between the body's own cells (self) and foreign invaders (non-self). Sometimes the immune system's recognition apparatus becomes misdirected and the body begins to mount an immune response directed against its own cells and organs. These misguided T cells and autoantibodies cause what are referred to as autoimmune diseases, which are a varied group of more than 80 serious, chronic illnesses that affect many human organ systems and tissues. For example, T cells that attack pancreas cells contribute to diabetes, while autoantibodies are common in people with rheumatoid arthritis. In another example, patients with systemic lupus erythematosus have antibodies to many types of their own cells and cell components. The treatment of autoimmune diseases depends on the type of disease, how severe it is and the symptoms. Therefore, the treatment may vary from relieving symptoms to preserving organ function (e.g., insulin injections to regulate blood sugar in diabetics) to targeting disease mechanisms (e.g., immunosuppressive drugs or immunomodulators).

Immunosuppression is also used to suppress alloimmune responses to transplantation antigens, i.e., host-versus-graft and graft-versus-host diseases. Alloimmune responses can determine the success or failure of three major transplant events—engraftment of transplanted organs, graft-versus-host disease (GVHD) and graft-versus-malignancy (GVM) effect. For tissue engraftment, e.g., organ transplantation, immunosuppression of the host immune system permits the transplant to avoid immune rejection. In the case of bone marrow transplantation, immunosuppression of the recipient is needed to allow the graft to gain a foothold. Recipients that do not achieve early donor T cell engraftment are at risk for graft rejection from residual host immune cells (Childs et al., Blood 1999, 94:3234). The direct (contacting antigen presenting cells) or indirect (cytokine induction) expansion of T cells recognizing recipient antigens (alloantigens) leads to tissue damage and GVHD (Ferrara and Deeg, N. Engl. J. Med. 1991, 324:667). GVM is an expansion of transplanted T cells in the bone marrow, but directed against malignant recipient cells, which is a beneficial effect.

Several immunosuppressive compounds exist to combat transplantation rejection, which include, for example, cyclosporine, steroids and methotrexate. However, side effects are associated with each of these drugs, such as kidney toxicity or more rarely neurological problems associated with cyclosporin; weight gain, irritability, and mood swings associated with steroids; and upset stomach, mouth sores, low white blood counts and liver and bone marrow toxicity associated with methotrexate. Attempts to minimize or eliminate GVHD prior to transplantation or transfusion by removing (e.g., with antibodies or by physical separation) or inactivating (e.g., irradiation) donor T cells were unsuccessful because there was an increased risk of rejection, relapse and infectious complications (Horowitz et al., Blood. 1990, 75:555).

Natural immune suppression, or immune regulation, is also known to occur. Immune regulatory cells of myeloid origin have been found in normal adult bone marrow of humans and animals (Schmidt-Wolf et al., Blood. 1992, 80:3242; Maier et al., J. Immunol. 1989, 143:4914; Sugiura et al., Proc. Natl. Acad. Sci. USA. 1988, 85:4824; Angulo et al., J. Immunol. 1995, 155:15), as well as in sites of intense hematopoiesis, such as in the spleen of newborn mice and during GVHD in adult mice, or following cyclophosphamide injection or γ-irradiation (Strober, Ann. Rev. Immunol. 1984, 2:219; Young et al., J. Immunol. 1997, 159:990). Down-regulation of T cell responses is associated with progressive tumor growth, and myeloid suppressor cells (MSCs; recently renamed myeloid derived suppressor cells or MDSC) were found to be involved in these negative immunoregulatory responses (Apolloni et al., J. Immunol. 2000, 165:6723-6730; Bronte et al., J. Immunol. 1998, 161:5313-5320).

In particular, tumor growth is accompanied by an increase in the number of Gr-1⁺/Mac-1⁺ (CD11b/CD18) Gr-1⁺/CD11b⁺ MSCs with strong immune suppressive activity in bone marrow (BM) and peripheral lymphoid organs in cancer patients (Young et al., J. Immunol. 1997, 159:990; Kusmartsev et al., Int. J. Immunopathol. Pharmacol. 1998, 11:171; Almand et al., J. Immunol. 2001, 166:678), and in tumor-bearing mice (Young et al., Cancer Res. 1987, 47:100; Subiza et al., Int. J. Cancer. 1989, 44:307; Kusmartsev et al., Exp. Oncology. 1989, 11:23; Young et al., J. Immunol. 1996, 156:1916). MSCs are capable of inhibiting the T cell proliferative response induced by alloantigens (Schmidt-Wolf et al., Blood. 1992, 80:3242; Brooks et al., Transplantation. 1994, 58:1096), CD3 ligation (Kusmartsev et al., J. Immunol. 2001, 165:779), and various mitogens (Schmidt-Wolf et al., Blood. 1992, 80:3242; Maier et al., J. Immunol. 1989, 143:4914; Sugiura et al., Proc. Natl. Acad. Sci. USA. 1988 85:4824; Angulo et al., J. Immunol. 1995, 155:15). MSCs can also inhibit interleukin-2 (IL-2) utilization by NK cells (Brooks et al., Transplantation. 1994, 58:1096) and NK cell activity (Kusmartsev et al., Int. J. Immunopathol. Pharmacol. 1998, 11:171).

MSC-mediated T cell inactivation in vitro has also been reported (Bronte et al., J. Immunol. 2003, 170:270-278; Rodriguez et al., J Immunol. 2003, 171:1232-1239; Bronte et al., Trends Immunol. 2003, 24:302-306; Kusmartsev et al., J. Immunol. 2004, 172: 989-999; Schmielau and Finn, Cancer Res. 2001, 61:4756-4760; Almand et al., J. Immunol. 2001, 166:678; Kusmartsev et al., J. Immunol. 2000, 165:779; Bronte et al., J. Immunol. 1999, 162:5728). For example, Gr-1⁺ MSCs were described for the treatment of autoimmune diabetes (Steptoe et al., Diabetes. 2005, 54:434-442); however, more effective treatments are necessary.

To date there are no methods for treating or preventing autoimmune diseases or alloimmune reactions that do not have undesirable side-effect profiles. Therefore, there remains a need for a method to treat or prevent autoimmune disease or alloimmune reactions while preserving a GVM effect, and at the same time does not cause severe side effects. The instant invention fills such a need and provides other related advantages.

### SUMMARY OF THE INVENTION

The present invention provides a method of treating an autoimmune disease or alloimmune response in an individual. The method comprises administering a therapeutically effective amount of myeloid suppressor cells (MSCs) to the individual, wherein the MSCs have a Gr-1⁺/CD11b⁺ phenotype. In one embodiment, the autoimmune disease is type I diabetes. In one embodiment, the alloimmune response is graft rejection or graft-versus-host disease (GVHD).

In another embodiment of the present invention, the MSCs are autologous. In another embodiment, the method further comprises administering an inhibitor of MSC terminal differentiation, which may be GM-CSF, M-CSF, or IL-3. In another embodiment, the method further comprises altering SHIP (SRC-homology-2-domain-containing inositol-5-phosphatase) signaling, increasing F4/80 expression, or administering one or more autoantigens. In another embodiment, the MSCs are genetically engineered to express or overexpress one or more autoantigens. In another embodiment, the method further comprises administering a cytokine to enhance suppression of anti-tumor responses and the development of Treg cells mediated by MSC. These cytokines may be IFN-γ, IL-10 or TGF-β. In another embodiment, the method further comprises administering an immunosuppressive drug, which may be cyclosporin, methotrexate, cyclophosphamide or tacrolimus. In another embodiment, the MSC phenotype further comprises CD115 or F4/80 cell surface markers. In another embodiment, the phenotype of the MSC includes at least one additional marker selected from CD31, c-kit, VEGF-receptor, or CD40. In another embodiment, the MSCs are recombinant MSCs modified to overexpress Gr-1, CD115, or F4/80.

The present invention also provides a method of producing myeloid suppressor cells (MSCs), which method comprises culturing primary hematopoietic stem cells (HSCs) in the presence of stem-cell factor (SCF) in an amount and for a time sufficient to allow HSCs to differentiate into MSCs, wherein the MSCs have a Gr-1⁺/CD11b⁺ phenotype. In another embodiment, the MSC phenotype further comprises CD115 or F4/80. In another embodiment, the phenotype includes at least one additional marker selected from CD31, c-kit, VEGF-receptor, or CD40. In another embodiment, the HSCs are recombinant HSCs modified to overexpress Gr-1, CD115, or F4/80. In another embodiment, the HSCs are further cultured in the presence of GM-CSF, M-CSF, G-CSF, Flit-3 ligand, or tumor-conditioned medium, or are genetically modified to express SCF, GM-CSF, M-CSF, Flit3 ligand or G-CSF. In another embodiment, the method provides for isolation of the MSCs, which may be by gradient centrifugation.

These and other aspects of the invention will be better understood by reference to the following drawings, Detailed Description, and Examples.

### BRIEF DESCRIPTION OF THE DRAWINGS

FIG. 1 shows flow cytometry dot-plots that demonstrate sorted Gr-1⁺ cells inhibited the proliferation of CD4⁺ T cells. The dot-plots were gated on CD4⁺ cells.

FIG. 2 shows cytokine and NO secretion measured by ELISA and Greiss reagent, respectively.

FIG. 3 shows induction of Foxp3⁺ T regulatory cell by Gr-1⁺/CD115⁺ MSC by assessing total RNA isolation and the expression of Foxp3 by RT-PCR (upper panel) and real-time PCR (lower panel).

FIG. 4 is a bar graph that shows the suppressive activity of Thy1.2 T cells co-cultured with CD4⁺ HA-specific TCR splenocytes at various ratios in the presence of HA peptide.

FIG. 5 shows the proliferation and Foxp3 expression levels of sorted T cells. The proliferative responses of adoptive sorted T cells from anti-IFN-γ and anti-IL-10 groups against HA peptide are significantly higher than those from the control Ig group (*p<0.01, ANOVA test).

FIG. 6 is a bar graph that shows the tumor weight of animals in the anti-IFN-γ and anti-IL-10 groups is significantly lower than that of those in the control Ig group (*p<0.01, ANOVA).

FIG. 7 shows TGF-β1, iNOS, and arginase1 gene expression in tumor tissues.

FIG. 8 is a bar graph that shows the proliferative response against HA or OVA peptide. Data (mean±standard deviation) are expressed as stimulation index (SI).

FIG. 9 shows gene expression of IL-10, TGF-β, arginase 1, and iNOS by sorted MSCs.

FIG. 10 is a bar graph showing ELISA of secreted IL-10 and TGF-β from sorted Gr-1⁺/CD115⁺ MSC with or without IFN-γ stimulation (*p<0.05 compared to unstimulated group, student's t-test).

FIG. 11 shows flow cytometry dot-plots showing an increase of Gr-1⁺/CD115⁺/F4/80⁺ cell population in BM and spleen Fr. 2 from tumor bearing animals. Gr-1 gated dot plots are presented and suppressive activity of Percoll Fr. 2 cells correlates with Gr-1 and CD115 markers.

FIG. 12 shows graphs of HA peptide-mediated HA CD4 TCR splenocyte proliferation responses.

FIG. 13 shows flow cytometry dot-plots gated on Gr-1 (upper panel) and the suppression of MSC on CD4⁺ HA-specific TCR splenocytes (lower panel).

FIG. 14. The sorted cells showed Foxp3 expression by RT-PCR and proliferative activity (*P<0.01, ANOVA TEST). Stimulation index (SI) was calculated by dividing the proliferation count (cpm) in the presence of HA peptide by that in the absence of HA peptide. Data shown, representative of two reproducible experiments, are mean values and standard deviations from three individual animals and the residual tumor weight from each group was measured

FIG. 15. Depletion of CD4⁺ CD25⁺ Treg enhances the tumor regression and proliferation response.

FIG. 16 shows tumor weights corresponding to administration of MSC, CD4 T cells, CD8 T cells, anti-CD25, and rat Ig (upper panel, **P<0.01 and *P<0.05 compared to the group without CD25 depletion and only T cell and MSC transfer, ANOVA). CD4 and CD8 T cells were recovered from spleen and stimulated with CD4 or CD8 HA-peptide for proliferative responses (lower panel).

FIG. 17 is a graph that shows iNOS was not required for the development of Treg cells in vivo. Data are presented as mean±standard deviation of triplicate cultures.

FIG. 18. The expression of Foxp3 and GAPDH in in vitro MSCs were analyzed by RT-PCR.

FIG. 19. The expression of Foxp3 and GAPDH in in vivo MSCs were analyzed by RT-PCR.

FIG. 20 is a bar graph showing proliferative response of tumor-specific T cells recovered from recipient tumor-bearing mice (*p<0.01, ANOVA test).

FIG. 21. Diabetes onset is suppressed by transfer of MSC with autoantigen. Ins-HA/RAG−/− mice were injected with 1×105 CD4 HA TCR T cells through tail vein. 24 hours later, the mice were injected with PBS or with 5×106 sorted MSC with HA peptide (5 μg) (n=19) or control peptide, OVA (5 μg) (n=6) or control Fr.3 cells+HA peptide (n=8) or left untreated (n=6) for a total of two doses every other day through tail vein. Blood glucose was measured by blood glucose meter (Bayer). The results are combined from two separate experiments.

FIG. 22. HE and Immunohistochemical analysis of insulin and β-islets of treated mice. Serial sections of pancreas were prepared from treated mice 4 weeks after the cell therapy. Sections were stained with HE (up panels) and stained with rabbit polyclonal anti-insulin (Santa Cruz Biotechnology, Inc.) followed by goat anti-rabbit Ig-HRP (Southern Biotech) and color development with substrates (Lower panels). Left panel: diabetic mice that were treated with T cell transferred alone. Right panel: non-diabetic mice that were treated with MSC+HA peptide.

FIG. 23. CD4 Immunohistochemical analysis of islets treated mice. Serial frozen sections of pancreata were prepared from treated mice 4 weeks after the cell therapy. Sections were incubated with anti-CD4 and co-stained with goat anti-mouse Ig-HRP (Southern Biotech) and color development with substrates. Right panel: diabetic mice that were treated with T cells transferred alone. Left panel: non-diabetic mice that were treated with MSC+HA peptide.

FIG. 24A. HA-mediated proliferation of autoreactive T cells recovered from treated mice. T cells were recovered from recipient Ins-HA RAG−/− mice 30 days after treatment and cultured in the presence of HA peptide (5 μg/ml). [3H]-Thymidine (1 μCi/well) was added for the last 8 hr of 72-hr culture. Stimulation index is calculated as cpm in the presence of HA divided by cpm in the absence of HA.

FIG. 24B. HA-mediated proliferation of autoreactive T cells recovered from treated mice. T cells were recovered from recipient Ins-HA RAG−/− mice 30 days after treatment and cultured in the presence of HA peptide (5 μg/ml). The cultured supernatant were harvested and measured for the cytokine e.g. IFNg, IL-10 and TGFb by ELISA (R&D Inc.).

FIG. 25A. Foxp3 gene expression in T cells recovered from treated mice. Total RNA was prepared from T cells recovered (by MACS) from non-diabetic mice that received transfer of MDSC+HA (Lane 1), diabetic mice that received transfer of MDSC+HA (Lane 2) or MDSC+OVA (Lane 3) or Fr.3 cell+HA (Lane 4), or CD4-HA-TCR T cell alone mice (Lane 5). Foxp3 or internal control GAPDH gene expressions were assessed by one-step RT-PCR using specific primer pairs.

FIG. 25B. Foxp3+CD4+CD25+ T cells in treated mice. Splenocytes from various treatment groups were stained with anti-CD4-FITC+anti-CD25-APC or isotype matched control antibodies, followed by overnight permeation and intracellular staining with anti-Foxp3-PE per manufacturer's instruction (eBioscience). CD4 gated dot plots are presented.

FIG. 26. CD25+ T cells mediated suppression. CD25+ T cells isolated from diabetes free mice were co-cultured with Thy1 purified T cells from HA TCR transgenic T cell at various ratio in the presence of HA peptide (μg/ml) and irradiated splenocyte as APC. [3H]-Thymidine (1 μCi/well) was added for the last 8 hr of 72-hr culture.

FIG. 27. MHC Class II expression on MSC is required for MSC mediated tumor specific T cells immune suppression in vivo. Reduction in Foxp3 expression by T cells recovered from mice that received MHC Class II KO MSC. Foxp3 gene expression was assessed by real time RT-PCR on total RNA prepared from the same number of sorted T cells. Intracelluar staining of Foxp3 gene expression were perform by e-bioscience kits.

FIG. 28 shows expression of SCF, VEGF, and BAFF by MCA26 tumor tissue and various murine and human tumor cell lines from multiple tissue origins.

FIG. 29 shows the effect of stem cell factor (SCF) on the accumulation of MSCs. (A) Establishment of stable SCF mRNA knockdown MCA26 clone A. (B) Proliferative responses of T cells in tumor tissues. (C) Decrease of Gr-1⁺/CD11b⁺/CD115⁺ MSC in bone marrow (BM).

FIG. 30 shows MSCs derived from primary bone marrow cells in the presence of SCF. (A) Gr-1⁺/CD11b⁺/CD115⁺ MSC derived from the culture of bone marrow cells in the presence of SCF. (B) Suppressive activity of in vitro derived MSCs. Percent suppression is calculated by ((cpm in the absence of MSC)−(cpm in the presence of MSC))/(cpm in the absence of MSC).

FIG. 31. MSC mediated suppression and Treg induction in mixed lymphocyte reaction. The purified BABL/c T cells were co-cultured with irradiated B6 splenocytes in the presence of Gr-1⁺ MSC from Percoll Fr. 2 or the control cells from Fr. 3 for six days. Suppressive activity and Foxp3 expression were analyzed by [³H]-thymidine incorporation assay and RT-PCR, respectively.

FIG. 32. MSC suppressed GVHD. Irradiated BALB/c mice were injected with T cell-depleted bone marrow cells (TCD-BM, 5×10⁶/mouse) from C57BL/6 mice, TCD-BM and column enriched splenic T cells from C57BL/6 (5×10⁵/mouse), or TCD-BM+column enriched splenic T cells+MSC (5×10⁶/mouse) from C57BL/6 (5×10⁵/mouse). T-cell depletion of bone marrow cells was performed twice by staining with anti-Thy-1 Ab conjugated with magnetic microbeads followed by MACS column. Depletion efficiency (99.8%) was confirmed by flow cytometry.

FIG. 33. Proliferative response of T cells recovered from treated mice. The sorted donor T cells from long-term surviving treated mice and anti-CD3 mediated proliferation was tested. 1×10⁵ T cells isolated from mice that received BM+T cells or BM+T cells+MSC were stimulated antiCD3 antibody (1 μg/ml) for 72 hours. [³H]-Thymidine was added for the last 8 hours of co-culture. The mean of cpm±standard deviation is presented.

FIG. 34. CD4⁺CD25⁺Foxp3⁺ T cells in treated mice. Splenocytes from treated mice were stained with anti-CD4-FITC+anti-CD25-APC or isotype control followed by permeation and staining with anti-Foxp3-PE or isotype control per manufacture's instruction (eBioscience). Dot plots gated on CD4⁺ cells are presented.

FIG. 35. Immunostaining of long term survival from controls of BM, or BM+MSC or experimental BM+T cell+MSC adoptive transferred mice by FACS analysis. The blood leukocytes were isolated and co-stained with CD4-PE, H-2 Kd-FITC and H-2 Kb-PE-Cy7, the results are gated on CD4 positive cells (top panel) or CD8-PE, H-2 Kd-FITC and H-2 Kb-PE-Cy7, the results are gated on CD8 positive cells (bottom panel).

FIG. 36 shows dot-plots demonstrating the T cell profile of recipient mice.

FIG. 37A. Left Panel: Expression of SCF by various murine and human tumor cell lines from multiple tissue origins. Total RNAs were prepared from various mouse (Colon cancer MCA26, MC38, Breast cancer JC, 4T1, Melanoma B16) and B) human tumor cell lines (colon cancer: HCT15, SW620, DLD-1, Colo205; breast cancer: MDA-MB435). The total RNAs were used to assess the expression of SCF by RT-PCR with specific primer pairs. C). Stable SCF mRNA knockdown MCA26 clone. MCA26 cells were stably transformed with an SCF siRNA expressing vector or a control vector. The expression of SCF was assessed by RT-PCR.

FIG. 38. Proliferative responses of T cells isolated from tumor tissues after anti-ckit blocking. Tumor infiltrating lymphocytes (TILs) were isolated from control rat Ig or various dose of anti-ckit treated MCA26 tumor bearing animals. The anti-CD3/anti-CD28 mediated proliferative responses of the T cells were assessed in a standard [³H]-thymidine incorporation assay. Splenic T cells purified from naïve mice were used as positive control. SI: stimulation index.

FIG. 39. Anti-ckit prevents the development of T-cell anergy in tumor bearing mice. Thy1.2⁺ CD4 HA-specific TCR-transgenic T cells (5×10⁶/mouse) were injected via tail vein into congenic Thy1.1⁺ MCA26 tumor-bearing mice and HA-MCA26 tumor-bearing mice three days after the first dose of anti-ckit or rat-Ig injection (50 mg/mouse). At day 15 after transfer, Thy1.2⁺ splenocytes were recovered by sorting. (A) Proliferative responses of sorted Thy1.2⁺ CD4 HA-specific T cells to HA peptides. The culture was pulsed with [³H]-thymidine for the last 8 hrs of 72-hr culture. Stimulation index (SI) is calculated as the proliferation count (cpm) in the presence of HA peptide divided by that in the absence of HA peptide. Data shown are representative of two reproducible experiments. (B) The residual tumor weight. The residual tumors were isolated and the tumor weight was measured. (C) The expression of Foxp3 in tumor-specific (CD4 HA TCR transgenic) T cells. RNA was prepared from Thy1.2⁺ CD4 HA TCR transgenic T cells recovered from treated mice and Foxp3 expression was analyzed by one-step RT-PCR and real-time RT-PCR. GAPDH expression was used as house keeping gene control. (D) Intracellular staining of Foxp3 in tumor-specific (CD4 HA TCR transgenic) T cells. Splenocytes were prepared from treated mice and stained with fluorochrome-conjugated anti-Thy1.2 (FITC)+anti-CD4 (APC)+anti-CD25 (PE-Cy7)+anti-Foxp3 (PE). Thy1.2⁺ CD4⁺ gated dot plots are presented. Left panel: naïve mice with adoptively transferred T cells and rat Ig; middle panel: tumor bearing mice with adoptively transferred CD4 T cells and rat Ig control; right panel: tumor bearing mice with adoptively transferred T cells and anti-ckit (E) Cytokine expression in tumor-specific T cells. Culture supernatants of recovered Thy1.2⁺ CD4 HA TCR transgenic T cells in the presence of HA peptide (5 mg/ml) and irradiated antigen presenting cells (naïve splenocytes) were collected. The naïve CD4 HA TCR splenocyte stimulated with and without HA peptide were used as positive and negative control separately. The cytokines, IFN-g, IL-12 (p70), IL-4, IL-10, and TGF-b concentrations were measured by ELISA kits (R&D Systems).

FIG. 40. The c-Kit mediated MSC accumulation and anti-angiogenesis. A) Immunostaining of tumor tissue with anti-mouse Gr-1-Cy3 antibody on the tumor tissue from wild-type (WT) MCA26 or SCF silenced MCA26 cells. B) Blocking SCF function by anti-c-kit treatment can prevent both MSC accumulation and blood vessel formation. BALB/c mice were intrahepatically inoculated with HA-MCA26 tumor cells. At day 9, mice were transferred with 5×10⁶ HA-TCR-T cells and injected with anti-c-kit (50 μg) or rat Ig as a control for four times every three days. One group of mice received no T cells but treated with rat Ig. The Gr-1 biotin and avidin Cy3 were used for staining of MSC as shown in C. The ant-iCD31-Cy3 antibody was used for immunostaining for blood vessel as shown in D. C,D) Similar profile of immunostaining for the blood vessel with anti-mouse CD31-Cy3 antibody in the wild-type (WT) MCA26 or SCF silenced MCA26 cells (in C) and control Ig or HA-TCR T cell alone or anti-ckit and HA-TCR T cell adoptive transferred mice (in D).

FIG. 41. Anti-ckit significantly improves the long-term survival rate of mice treated with immune modulatory therapy of IL-12+4-1BB activation. Mice bearing large MCA26 tumors (10×10 mm²) in liver were divided into the following treatment groups: (1) control viral vector DL312+control Ig (solid circle); (2) DL312+anti-ckit (solid square); (3) Adv.mIL-12+anti-4-1BB+rat Ig (open circle); (4) Adv.mIL-12+anti-4-1BB+anti-ckit (open square). P<0.001, by logrank survival analysis.

FIG. 42. Fr. II CD115+/F4/80+ cells are also IL-4 receptor positive.

### DETAILED DESCRIPTION

The present invention relates to myeloid suppressor cells (MSCs), their production, and their use in treating autoimmune diseases and alloimmune responses. In certain embodiments, MSCs can be produced by culturing primary hematopoietic stem cells (HSCs) in the presence of, for example, stem-cell factor (SCF) and SCF with condition medium from tumor factors, e.g. (M-CSF, GM-CSF, IL-3, Flt-3 ligand). In further embodiments, where desired, a T cell response can be reduced and T regulatory cells (Tregs) can be induced upon administration of MSCs. For example, type I diabetes (T1D) and graft-versus-host disease (GVHD) can be prevented or treated by administration of these MSCs.

More specifically, the present invention provides MSCs that can suppress the antigen specific immune response of autoactivated T cells against islet cells, thereby treating type I diabetes.

In still other embodiments, T cell tolerance can be produced through administration of these MSCs.

In still further embodiments, the present invention provides a method for significantly increasing the concentration of MSCs, such as of Gr-1⁺/CD11b⁺, Gr-1⁺/CD11b⁺/CD115⁺, and Gr-1⁺/CD11b⁺/F4/80⁺ MSCs, wherein the method used to isolate MSCs is a Percoll density gradient from bone marrow cells and splenocytes. For example, the Percoll density gradient fraction 2 (Fr. II; 1.063-1.075 g/ml) contains such MSCs. These MSC cells not only have the ability to strongly inhibit anti-CD3/anti-CD28 mediated proliferation of naïve T cells but also play an important role in the suppression of the T cell immune response against malignancies. In other embodiments, MSCs can be used in combination with other immunosuppressive therapies, such as methotrexate, monoclonal antibodies against antigens expressed on mature T cells, corticosteroids, and antithymocyte globulin (ATG).

The proteins described herein are known by several names. The table below outlines these.

| Name | Synonyms |
|---|---|
| Gr-1 | Lymphocyte antigen Ly-6G.1 precursor (Ly6g) |
| F4/80 | Cell surface glycoprotein EMR1, Cell surface glycoprotein |
| | F4/80, DD7A5-7, EGF-TM7, EMR1 hormone receptor, F4/80, |
| | Gpf480, Ly71, TM7LN3 |
| CD11b | CD11b/CD18, Cell surface glycoprotein MAC-1 alpha subunit, |
| | CR3 alpha |
| | chain, F730045J24Rik, integrin alpha M, Leukocyte adhesion |
| | receptor MO1, Ly-40, Mac-1, Mac-1a |
| CD115 | Macrophage colony stimulating factor I receptor precursor |
| | (CSF1R), colony stimulating factor 1 receptor (c-fmsr), |
| | Fim-2, Fms, Fms proto-oncogene |
| SCF | Kitl, Kit ligand precursor, C-kit ligand, Clo, Con, |
| | contrasted, Gb, Hematopoietic growth factor KL, Kitlg, kit |
| | ligand, Mast cell growth factor, MGF, SCF, SF, Sl, SLF, |
| | Steel, Steel factor, Stem cell factor |
| BAFF | B-cell activating factor, also known as BLyS, TALL-1, |
| | THANK, zTNF4, TNFSF13B |

### DEFINITIONS

The term “myeloid suppressor cell (MSC)” refers to a cell that is of hematopoietic lineage and expresses Gr-1 and CD11b; MSCs are also referred to as immature myeloid cells and were recently renamed to myeloid-derived suppressor cells (MDSCs). MSCs may also express CD115 and/or F4/80 (see Li et al., Cancer Res. 2004, 64:1130-1139). MSCs may also express CD31, c-kit, vascular endothelial growth factor (VEGF)-receptor, or CD40 (Bronte et al., Blood. 2000, 96:3838-3846). MSCs may further differentiate into several cell types, including macrophages, neutrophils, dendritic cells, Langerhand cells, monocytes or granulocytes. MSCs may be found naturally in normal adult bone marrow of human and animals or in sites of normal hematopoiesis, such as the spleen in newborn mice. Upon distress due to graft-versus-host disease (GVHD), cyclophosphamide injection, or γ-irradiation, for example, MSCs may be found in the adult spleen. MSCs can suppress the immunological response of T cells, induce T regulatory cells, and produce T cell tolerance. Morphologically, MSCs usually have large nuclei and a high nucleus-to-cytoplasm ratio. MSCs can secrete TFG-β and IL-10 and produce nitric oxide (NO) in the presence of IFN-γ or activated T cells. MSCs may form dendriform cells; however, MSCs are distinct from dendritic cells (DCs) in that DCs are smaller and express CD11c; MSCs do not express CD11c. MSCs can be isolated as described, e.g., in the Examples. T cell inactivation by MSCs in vitro can be mediated through several mechanisms: IFN-γ-dependent nitric oxide production (Kusmartsev et al. J Immunol. 2000, 165: 779-785); Th2-mediated-IL-4/IL-13-dependent arginase 1 synthesis (Bronte et al. J Immunol. 2003, 170: 270-278); loss of CD34 signaling in T cells (Rodriguez et al. J Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

The term “primary hematopoietic stem cell (HSC)” refers to a cell that can give rise to all blood and lymphoid cell types including, for example, red blood cells, platelets, white blood cells, MSCs, B cells, and T cells. HSCs can also propagate themselves, i.e., give rise to other HSCs, and may give rise to non-hematological cell types. HSC also have a long term reconstitution ability. HSCs are large cells that express Sca-1 and c-kit, have a high nucleus-to-cytoplasm ratio, and may express CD34.

Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Recent studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A “primary immune response” refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) communicate with these T cells, leading to the differentiation of naive CD4+ T cells into T-helper 1 (Th1) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A “secondary immune response” or “adaptive immune response” may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response

An “autoimmune disease” or “autoimmune response” is a response in which the immune system of an individual initiates and may propagate a primary and/or secondary response against its own tissues or cells. An “alloimmune response” is one in which the immune system of an individual initiates and may propagate a primary and/or secondary response against the tissues, cells, or molecules of another, as, for example, in a transplant or transfusion.

The term “cell-mediated immunity” refers to (1) the recognition and/or killing of virus and virus-infected cells by leukocytes and (2) the production of different soluble factors (cytokines) by these cells when stimulated by virus or virus-infected cells. Cytotoxic T lymphocytes (CTLs), natural killer (NK) cells and antiviral macrophages are leukocytes that can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions.

A host T cell response can be directed against cells of the host, as in autoimmune disease. For example, the T cells in type I diabetes (T1D) recognize an “antigen” that is expressed by the host, which causes the destruction of normal host cells—for T1D, the endocrine β-cells of the islets of Langerhans of the pancreas. A T cell response may also occur within a host that has received a graft of foreign cells, as is the case in graft-versus-host disease (GVHD) in which T cells from the graft attack the cells of the host, or in the case of graft rejection in which T cells of the host attack the graft.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10- (IL-10-) secreting T regulatory type 1 (Tr1) cells, transforming growth factor-β- (TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

The phrase “inducing T regulatory cells” means activating Tregs to inhibit or reduce the T cell response. One method of induction is through the use of the MSCs of the present invention.

The phrase “T cell tolerance” refers to the anergy (non-responsiveness) of T cells when presented with an antigen. T cell tolerance prevents a T cell response even in the presence of an antigen that existing memory T cells recognize.

The term “differentiate” refers to the genetic process by which cells are produced with a specialized phenotype. A differentiated cell of any type has attained all of the characteristics that define that cell type. This is true even in the progression of cell types. For example, if cell type X matures to cell type Y which then overall matures to cell type Z, an X cell differentiates to a Y cell when it has attained all of the characteristics that define a type Y cell, even though the cell has not completely differentiated into a type Z cell.

The term “SHIP” refers to (SRC-homology-2-domain-containing inositol-5-phosphatase). SHIP catalyzes the hydrolysis of the membrane inositol lipid PIP3, thereby preventing activation of PLCγ and Tec kinases and abrogating the sustained calcium flux mediated by the influx of calcium through the capacitance coupled channel. SHIP signaling is known to affect maturation of MSCs (Ghansah et al. J. Immunol. 2004, 173:7324-7330).

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

“Cytokine” is a generic term for a group of proteins released by one cell population which act on another cell population as intercellular mediators. Examples of such cytokines are lymphokines, monokines, and traditional polypeptide hormones. Included among the cytokines are interferons (IFN, notably IFN-γ), interleukins (IL, notably IL-1, IL-2, IL-4, IL-10, IL-12), colony stimulating factors (CSF), macrophage colony stimulating factor (M-CSF), granulocyte macrophage colony stimulating factor (GM-CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), osteoinductive factors, etc. Those of particular interest for the present invention include IFN-γ, IL-10, and TGF-β.

“Autoantigen” refers to a molecule that is endogenous to a cell or organism that induces an autoimmune response.

“Transplant rejection” means that a transplant of tissue or cells is not tolerated by a host individual. The transplant is not tolerated in that it is attacked by the host's own immune system or is otherwise not supported by the host. The transplant may be an allotransplant, a transplant of tissue or cells from another individual of the same species, or an autotransplant, a transplant of the host's own tissue or cells. Transplant rejection encompasses the rejection of fluids through transfusion.

The term “subject” or “individual” as used herein refers to an animal having an immune system, preferably a mammal (e.g., rodent such as mouse). In particular, the term refers to humans.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

A “nucleic acid molecule” (or alternatively “nucleic acid”) refers to the phosphate ester polymeric form of ribonucleosides (adenosine, guanosine, uridine, or cytidine: “RNA molecules”) or deoxyribonucleosides (deoxyadenosine, deoxyguanosine, deoxythymidine, or deoxycytidine: “DNA molecules”), or any phosphoester analogs thereof, such as phosphorothioates and thioesters, in either single stranded form, or a double-stranded helix. Oligonucleotides (having fewer than 100 nucleotide constituent units) or polynucleotides are included within the defined term as well as double stranded DNA-DNA, DNA-RNA, and RNA-RNA helices. This term, for instance, includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

“Treating” or “treatment” of a state, disorder or condition includes:
(1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition,
(2) inhibiting the state, disorder or condition, i.e., arresting, reducing or delaying the development of the disease or a relapse thereof (in case of maintenance treatment) or at least one clinical or subclinical symptom thereof, or
(3) relieving the disease, i.e., causing regression of the state, disorder or condition or at least one of its clinical or subclinical symptoms.

The benefit to an individual to be treated is either statistically significant or at least perceptible to the patient or to the physician.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3^{rd} ed. Cold Spring Harbor Laboratory Press: Cold Spring Harbor, N.Y.; Sambrook et al., (1989) Molecular Cloning: A Laboratory Manual. 2^{nd} ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

### Generation of MSCs or Cells with MSC Functions

In another aspect, the present invention provides methods for the production of MSCs. In one embodiment, hematopoietic stem cells (HSCs) isolated from normal mouse can be stimulated to differentiate into Gr-1⁺/CD11b⁺, Gr-1⁺/CD11b⁺/CD115⁺, Gr-1⁺/CD11b⁺/F4/80⁺, or Gr-1⁺/CD11b⁺/CD115⁺/F4/80⁺ MSCs by culturing in the presence of stem-cell factor (SCF) or SCF with tumor factors, which can increase the MSC population. In further embodiments, other cytokines may be used, e.g., GM-CSF, M-CSF, G-CSF. Any one of the cytokines may be used alone or in combination with SCF or other cytokines. In still another embodiment, tumor-conditioned media may be used with or without SCF to stimulate HSCs to differentiate into MSCs. As used herein, “tumor-conditioned medium” is the supernatant of a tumor cell culture.

Another embodiment provides HSCs genetically engineered to produce antigens, which are required and specific for immune suppression. Methods of genetic engineering are well known to those of ordinary skill in the art.

In a further embodiment, a genetically engineered non-MSC cell can be generated to function similar to MSCs of the present invention with immune suppressive activity. For example, this may be achieved through the expression or overexpression of Gr-1, CD11b, CD115, and/or F4/80. Additionally, the addition of cytokines may facilitate the functioning of the engineered non-MSC to imitate the immune suppressive effects of MSCs.

Cells may be isolated by any one of several techniques known to those of ordinary skill in the art. One technique is centrifugation. The centrifugation may or may not be with the use of a gradient. The Examples section describes centrifugation of cells in the presence of a Percoll gradient. This technique separates cells based upon density. Another such technique that may be used is panning, as described in Example 1. This technique uses immobilized molecules, for example, antibodies, that recognize and bind to molecules on the surface of a cell. The immobilized molecules recognize and bind to one or more specific cell surface molecules of a particular cell type. Cells that possess the one or more cell surface molecules are bound by the immobilized molecules, allowing any other cell to be washed away, retaining only the cell type of interest. Another example includes fluorescence activated cell sorting (FACS). Antibodies with fluorescent tags may be used to bind to the cells of interest. The antibodies bind to the cell surface molecules, and a FACS sorter may then sort and collect the cells based upon the fluorescence observed. The cells that display certain fluorescence may then be isolated. Another method of isolation well known in the art includes the use of tagging cells, based on their cell surface markers, with magnetic beads and separating the cells through the use of a magnetic column, as described in the Examples section.

In one embodiment, the instant disclosure provides a method of producing myeloid suppressor cells (MSCs), which method comprises culturing primary hematopoietic stem cells (HSCs) in the presence of stem-cell factor (SCF) in an amount and for a time sufficient to allow HSCs to differentiate into MSCs, wherein the MSCs have a Gr-1⁺/CD11b⁺ phenotype. In other embodiments, the produced MSC cells are isolated, such as by gradient centrifugation.

### Pharmaceutical Compositions

The present invention provides for myeloid suppressor cells in pharmaceutical compositions. Pharmaceutical compositions can be prepared by mixing a therapeutically effective amount of the active substance with a pharmaceutically acceptable carrier that can have different forms, depending on the route of administration. Pharmaceutical compositions can be prepared by using conventional pharmaceutical excipients and methods of preparation. All excipients may be mixed with disintegrating agents, solvents, granulating agents, moisturizers and binders. Furthermore, anti-M-CSF or anti-CSF antibodies may be administered to prevent the MSC of the present invention from differentiating.

As used herein, the term “therapeutically effective amount” refers to an amount which results in measurable amelioration of at least one symptom or parameter of a specific disorder. A therapeutically effective amount of the compound of the present invention can be determined by methods known in the art. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

The phrase “pharmaceutically acceptable,” as used in connection with compositions of the invention, refers to molecular entities and other ingredients of such compositions that are physiologically tolerable and do not typically produce untoward reactions (such as gastric upset, dizziness and the like) when administered to a human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “pharmaceutically acceptable salts, esters, amides, and prodrugs” refers to those salts (e.g., carboxylate salts, amino acid addition salts), esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The term “carrier” applied to pharmaceutical or vaccine compositions of the invention refers to a diluent, excipient, or vehicle with which a compound (e.g., an antigen and/or an adjuvant comprising a compound of the invention) is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The pharmaceutical compositions and unit dosage forms of the present invention for parenteral administration, and in particular by injection, typically include a pharmaceutically acceptable carrier, as described above. A preferred liquid carrier is vegetable oil.

### Administration

The MSCs of the present invention can be administered to individuals through injection (for example, intravenous, epidural, intrathecal, intramuscular, intraluminal, intratracheal or subcutaneous), orally, transdermally, or other methods known in the art. Administration may be once a day, twice a day, or more often, but frequency may be decreased during a maintenance phase of the disease or disorder, e.g., once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on recession of pathological signs and clinical and subclinical symptoms of a disease condition or disorder contemplated for treatment with the present compounds.

Also, host MSCs may be cultured in the presence of host or graft T cells ex vivo and re-introduced into the host. This may have the advantage of the host recognizing the MSCs as self and better providing reduction in T cell activity.

Dosages and administration regimen can be adjusted depending on the age, sex, physical condition of administered as well as the benefit of the conjugate and side effects in the patient or mammalian subject to be treated and the judgment of the physician, as is appreciated by those skilled in the art.

An individual in need thereof is, for example, a human or other mammal that would benefit by the administration of the MSCs of the present invention.

It will be appreciated that the amount of MSCs of the invention required for use in treatment will vary with the route of administration, the nature of the condition for which treatment is required, and the age and condition of the patient and will be ultimately at the discretion of the attendant physician or veterinarian.

The MSCs described herein can be used to treat autoimmune diseases and alloimmune responses. For example, the MSCs described herein may be used for treating or preventing diseases that involve a T cell response, such as T1D, GVHD, multiple sclerosis, thyroiditis, rheumatoid arthritis, and the like.

### Methods of Treatment

The present invention provides for the use of myeloid suppressor cells to treat autoimmune diseases, alloimmune responses, or any other disease, disorder or condition that involves a T cell response. Generally, these are conditions in which the immune system of an individual (e.g., activated T cells) attacks the individual's own tissues and cells, or implanted tissues, cells, or molecules (as in a graft or transplant). Exemplary autoimmune diseases that can be treated with the methods of the instant disclosure include type I diabetes, multiple sclerosis, thyroiditis (such as Hashimoto's thyroiditis and Ord's thyroiditis), Grave's disease, systemic lupus erythematosus, scleroderma, psoriasis, arthritis, rheumatoid arthritis, alopecia greata, ankylosing spondylitis, autoimmune hemolytic anemia, autoimmune hepatitis, Behçet's disease, Crohn's disease, dermatomyositis, glomerulonephritis, Guillain-Barré syndrome, inflammatory bowel disease, lupus nephritis, myasthenia gravis, myocarditis, pemphigus/pemphigoid, pernicious anemia, polyarteritis nodosa, polymyositis, primary biliary cirrhosis, rheumatic fever, sarcoidosis, Sjögren's syndrome, ulcerative colitis, uveitis, vitiligo, and Wegener's granulomatosis. Exemplary alloimmune responses that can be treated with the methods of the instant disclosure include graft-versus host disease, graft versus leukemia and transplant rejection.

In certain embodiments, the instant disclosure provides a method of treating an autoimmune disease or alloimmune response in an individual, which method comprises administering a therapeutically effective amount of myeloid suppressor cells (MSCs) to the individual, wherein the MSCs have a Gr-1⁺/CD11b⁺ phenotype. In another embodiment, the MSCs of this method are autologous. In still other embodiments, the method will further comprise administering an inhibitor of MSC terminal differentiation, such as inhibitors that block the activity of Flit3 ligand, GM-CSF, M-CSF, or IL-3. In yet another embodiment, the method will further comprise altering receptor signaling, such as the signaling of the SHIP receptor. In further embodiments, the method further comprises administering a cytokine, such as IFN-γ, IL-10 or TGF-β, or an immunosuppressive drug, such as cyclosporin, methotrexate, cyclophosphamide or tacrolimus.

Further description is provided with respect to T1D and GVHD as exemplary of the use of MSCs in the methods of the invention.

### Type I Diabetes

Type I diabetes (T1D), which affects one million Americans, is marked by a deficiency in endocrine β-cells in the pancreatic islets of Langerhans resulting from autoimmunity, which causes β-cell destruction by autoaggressive CD4 and CD8 T cells (Atkinson et al., N. Engl. J. Med. 1994, 331:1428; Von Boehmer et al., Science. 1999, 284:1135). Daily injection of insulin is the current treatment for T1D, but severe side effects develop over time because insulin injections cannot match the precise timing and dosing of physiological insulin secretion in response to hyperglycemia. Improper control of glucose levels in the blood results in hyperglycemia, which leads to chronic complications, such as widespread vascular damage with resulting kidney failure, blindness, heart disease, and chronic ulcers (Atkinson et al., N. Engl J. Med. 1994, 331:1428). For end-stage type I diabetic patients, there has been considerable progress recently in the use of pancreatic islet transplantation due to improvements in islet isolation, transplantation techniques, and immunosuppressive methods (Shapiro et al., Lancet. 2001, 358:Suppl:S21). Prevention of the onset of T1D will be aided by a treatment that induces antigen specific immune suppression against the autoimmune T cells and that prolongs the survival of islet transplants.

In T1D, also known as insulin-dependent diabetes mellitus (IDDM) or juvenile-onset diabetes, T cells of the individual's immune system attack its own β-cells. This reduces and eventually eliminates (when all of the β-cells are destroyed) insulin secretion into the blood stream. A decrease in insulin reduces the uptake of glucose by both hepatic and non-hepatic tissues. The blood glucose level remains high for a sustained amount of time, several hours longer than normal. This saturates the kidneys, which start to excrete excess glucose in urine. Due to the osmolytic nature of glucose, water is also excreted to balance the osmotic pressure across the nephrologic tissues. This leads to dehydration.

Currently, the treatment of T1D involves timed injections of insulin, but this treatment is only a substitute for organ function and does not target the disease mechanism. The instant disclosure provides methods for treating the cause of T1D, i.e. limiting the destruction of β-cells by the T cell response. As set forth above, in certain embodiments, the instant disclosure provides a method of treating type I diabetes in an individual, which method comprises administering a therapeutically effective amount of myeloid suppressor cells (MSCs) to the individual, wherein the MSCs have a Gr-1⁺/CD11b⁺ phenotype.

### Graft-Versus-Host Disease

In graft-versus-host disease (GVHD), the T cells of the donor bone marrow (BM) in a bone marrow transplant (BMT), or less commonly the T cells in a blood transfusion, develop an immune response against the cells of the host receiving the transplant or transfusion. The cell types most often attacked within the host are those of the skin, liver, and gut. GVHD is more likely to develop the more disparate the donor BM type is from the host BM type. Severity of disease is also correlated to disparity of BM type.

GVHD may be either acute or chronic. The acute form often first manifests as a skin rash but can quickly become life-threatening. Symptoms can include rash and other disorders of the skin, jaundice when the liver is affected, and bloody or watery diarrhea or cramps if the stomach is affected. Approximately 20-40% of those with GVHD die from the disease.

Currently, the treatment of GVHD involves the use of immunosuppressive drugs that cause a variety of unwanted side effects. The present disclosure provides methods for treating alloimmune responses with minimal side effects. As set forth above, in certain embodiments, the instant disclosure provides a method of treating GVHD in an individual, which method comprises administering a therapeutically effective amount of myeloid suppressor cells (MSCs) to the individual, wherein the MSCs have a Gr-1⁺/CD11b⁺ phenotype

A person of ordinary skill in the art may use well known molecular biology techniques to improve the function of the MSCs as described herein. The MSCs may be genetically engineered to endogenously express or overexpress antigen for T cell activation. Also, MSCs may be genetically engineered to express or overexpress CD115 and/or F4/80, for example, in Gr-1⁺/CD11b⁺ MSCs.

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

### EXAMPLES

### Example 1

### Isolation of Myeloid Suppressor Cells from Mice

Spleens, tibias, and femurs were harvested from mice under sterile conditions. Bone marrow (BM) cells were obtained by flushing the contents of the mouse femora and tibia with cold phosphate buffered saline (PBS) using a syringe and a 26-gauge needle. Spleen cell (SC) suspensions were prepared by teasing the spleen, which includes homogenization with two pieces of frosty cover slides, lysis of RBCs, and then passing the solution through a filter net. Isolated BM and SC were centrifuged for 5 minutes at 200×g and resuspended in complete culture medium (RPMI 1640 medium with 10% fetal calf serum (FCS), 20 mM HEPES buffer, 200 U/ml penicillin, 50 μg/ml streptomycin, 0.05 mM β-mercaptoethanol (2-ME), and 2 mM glutamine (all from Sigma, St. Louis, Mo.)).

Samples of BM or SC (3-4×10⁶ cells/ml) were placed in 75 cm² tissue culture flasks (Costar, Cambridge, Mass.) and incubated overnight at 37° C. in 5% CO₂. The next day, the nonadherent cells were recovered, washed and counted.

The isolated nonadherent cells were separated according to their density characteristics by centrifugation on a Percoll density gradient (described in Kusmartsev et al., J. Immunol. 2000, 165:779-785 and Angulo et al., J. Immunol. 1995, 155:15-26). Briefly, the recovered nonadherent cells (0.5-1×10⁸) were resuspended in 2 ml of 100% Percoll solution (Pharmacia). Two milliliters each of 70, 60, 50, and 40% Percoll and 1 ml of HBSS (Hank's Balanced Salt Solution) were carefully layered over the cell suspension. After centrifugation at 1800×g for 30 min, cells were collected from the gradient interfaces. Cells banding between 40 and 50% (<1.063 g/ml) were labeled as fraction (Fr.) I; between 50 and 60% (1.063-1.075 g/ml) as Fr. II (or Fr. 2); and between 60 and 70% (1.075-1.090 g/ml) as Fr. III. After washing, the cells were counted and adjusted to the appropriate concentrations in culture medium.

Myeloid cell-enriched Percoll fractions were depleted of Gr-1⁺/CD11b⁺ cells by panning (Wysocki et al., Proc. Natl. Acad. Sci. 1978, 75:2844-2848). Plastic petri dishes were each coated with 7 ml of secondary anti-rat IgG2b Abs (10 μg/ml; PharMingen, San Diego, Calif.). Fractionated BM or SC were incubated with primary Gr-1 Abs (PharMingen, San Diego, Calif.) in PBS without Ca²⁺/Mg²⁺ at a concentration 10 μg/10⁷ cells. After washing, the cells were plated onto the precoated petri dishes and incubated for 1 hr at 4° C. Nonadherent, Gr-1⁻ cells were then removed by gently washing with PBS. Coated microbeads or FACS was then used to further sort the cells based on CD115 and F4/80.

Fr. II cells were sometimes derived from the spleen of murine colon carcinoma MCA-26 tumor-bearing BALB/c mice in which these cells were also depleted of T cells by means of complement dependent lysis using anti-CD3 mAbs (PharMingen, San Diego, Calif.).

### Example 2

### T Cell Anergy and T Regulatory (Treg) Cell Development Mediated by MSCs

### Experimental Animals

10-week-old female congenic Thy-1.1⁺ BALB/c mice (Kemp et al. J. Immunol. 2004, 173:2923-2927) were a gift from Dr. Richard Dutton, (Trudeau Institute), and C57BL/6 mice were purchased from National Cancer Institute (Frederick, Md.). Influenza hemagglutinin (HA)-specific I-E^{d}-restricted CD4 and CD8 TCR-transgenic mice (in BALB/c background, Thy-1.2) were gifts from Dr. Linda Sherman (Scripps Research Inst., La Jolla, Calif.) and Dr. Constantin A. Bona (Mount Sinai School of Medicine, New York, N.Y.), respectively (see Marzo et al. Cancer Res. 1999, 59: 1071-1079; Morgan et al. J. Immunol. 1996, 157:978-983). Stat1 deficient BALB/c mice and IL-10R deficient mice were established as described before (Durbin et al. Cell. 1996, 84:443-450; Spencer et al. J. Exp. Med. 1998, 187:571-578). Mice deficient in inducible nitric oxide synthase (iNOS; in C57BL/6 background) or IL-4 receptor ax chain (IL-4Rα; in BALB/c background) and CD4 ovalbumin (OVA) specific TCR transgenic (OT II) C57BL/6 were purchased from the Jackson Laboratory (Bar Harbor, Me.). All animal experiments were performed in accordance with the animal guidelines of the Mount Sinai School of Medicine.

### Tumor Models

The MCA26 tumor cell line is a BALB/c-derived, chemically induced colon carcinoma line with low immunogenicity (Corbett et al. Cancer Res. 1975, 35:2434-2439). In order to establish a model in which tumor antigen-specific T cell responses can be tracked in vivo, the MCA26 colon tumor cell line was stably transformed with the gene encoding influenza hemagglutinin (HA) (a generous gift from Dr. Adolfo Garcia-Sastre, MSSM). The tumorigenicity of HA-transfected MCA26 (HA-MCA26), clone 44, was confirmed by implantation into syngeneic BALB/c mice. Similar in vivo tumor growth rates were observed for control neo plasmid-transfected parental MCA26 and clone 44 cells. The OVA-expressing tumor line used is an OVA-transfected clone derived from the murine B16 (H-2^{b}) melanoma (Mayordomo et al. Nat. Med. 1995, 1:1297-1302). To generate the tumor model of metastatic colon cancer, MCA26 or HA-MCA26 tumor cells (9×10⁴) were inoculated in the liver by intrahepatic implantation of cells as previously described (Kusmartsev et al. J. Immunol. 2000, 165:779-785). Similar methodology was used for the B16 tumor model.

### Peptide and Antibodies

CD4 HA peptide (¹¹⁰SFERFEIFPKE¹²⁰), CD8 HA peptide (⁵³³IYSTVASSL⁵⁴¹), and CD4 OVA peptide (³²³ISQAVHAAHAEINEAGR³³⁹) were purchased from Washington Biotechnology, Inc. (Baltimore, Md.). Neutralizing anti-mouse IL-10, IL-13, and IFN-γ antibodies were purchased from R&D Systems (Minneapolis, Minn.). Anti-Thy1.2-FITC, anti-Gr-1-APC or FITC, anti-CD115-PE, anti-F4/80-FITC, anti-CD11b-APC or FITC, anti-CD25-APC and isotype-matched mAbs were purchased from eBioscience (San Diego, Calif.).

### CFSE Labeling

Splenocytes from transgenic BALB/c mice were labeled with carboxy-fluorescein diacetate succinimidyl ester (CFSE, Molecular Probes, Eugene, Oreg.). Briefly, the cells were suspended in serum-free RPMI-1640 and incubated with CFSE (5 μM) at 37° C. for 10 min, followed by quenching with an equal volume of cold fetal calf serum and washing 3 times with complete medium and twice with cold PBS.

### Isolation of Fr.2 MSC

Mice with tumor sizes greater than 10×10 mm² were sacrificed and their spleen, tibias, and femurs were harvested. After lysis of red blood cells, bone marrow cells and splenocytes were fractionated by centrifugation on a Percoll (Amersham Biosciences, Sweden) density gradient as described (Example 1 and Kusmartsev et al. J. Immunol. 2000, 165:779-785). Cells were collected from the gradient interfaces. Cells banding between 40 and 50% were labeled as fraction 1 (Fr. 1); between 50 and 60% as Fr. 2; and between 60 and 70% as Fr. 3.

### Cell Sorting

In all of the sorting experiments, very stringent gating conditions were used (FACSVantage with FACSDiVa). The purity of the sorted cells was checked by flow cytometry and sorted cell populations that were greater than 97-98% pure MSC or T cells were chosen for the following experiments.

### MSC Suppression Assay

The suppressive activity of MSC was assessed in a peptide-mediated proliferation assay of TCR transgenic T cells as described previously (Li et al. Cancer Res. 2004, 64:1130-1139). Briefly, the splenocytes (1×10⁵) from TCR-transgenic mice were cultured in the presence of serial dilutions of irradiated MSCs in 96-well microplates. [³H]-thymidine was added during the last 8 h of 72-hr culture.

### Cytokine Detection by Enzyme-Linked Immunosorbent Assay (ELISA) and Nitric Oxide (NO) Measurement

Cytokine ELISAs were performed on culture supernatants using the mouse IL-2, IL-4, IL-10, IL-13, IFN-γ, and TGF-β ELISA kits (R&D Systems) per the manufacturer's instructions. Nitric oxide was measured by Greiss reagent (Sigma-Aldrich, St. Louis, Tex.)

### Mice Irradiation

Mice were irradiated with high dose radiation (850 rad) to eradicate endogenous MSC and T cells, which was confirmed by flow cytometric analysis of Gr-1⁺/CD115⁺ cells and T cells in bone marrow and spleen of irradiated mice which showed less than 0.5% of T cells and MSC were present in the recipient mice.

### Adoptive Transfer Experiments

Thy1.2 congenic CD4 or CD8 HA-specific TCR-transgenic T cells were enriched by T cell enrichment columns per manufacturer's instructions (R&D Systems) for adoptive transfer through tail vein injection (5×10⁶ cells/mouse). As for MSC, sorted Gr-1⁺/CD115⁺ bone marrow Fr.2 cells (2.5×10⁶/mouse) or single Gr-1⁺ Fr. 2 cells (5×10⁶/mouse) from large tumor-bearing mice was used.

9×10⁴ HA-MCA26 cells (or neo transfected parental MCA26 cells as a control) were inoculated into Thy1.1⁺ BALB/c mice. 6 days later, the mice with tumor size of around 5×5 mm² were irradiated. The following day, the sorted MSC and T cells were co-adoptively transferred through tail vein. Mice were sacrificed at day 7 after the adoptive transfer and Thy1.2⁺ T cells were recovered from spleen and lymph nodes of recipient mice by cell sorting.

### Proliferation Assay

The sorted Thy1.2⁺ or column enriched T cells (1×10⁴) with irradiated (2500 rad) naïve splenic cells (4×10³) as APC were co-cultured with or without HA peptide (5 μg/ml) in 96-well microplates. [³H]-thymidine was added during the last 8 hours of 72-hour culture.

### Reverse Transcription-PCR and Quantitative Real-Time PCR

Target cells were homogenized in TRIzol reagent (Invitrogen) and total RNA was extracted per manufacturer's instructions. An RT-PCR procedure was used to determine relative quantities of mRNA (One-step RT-PCR kit, Qiagen). Twenty-eight PCR cycles were used for all of the analyses. The intensity of each amplified DNA bands was further analyzed by IQ Mac v1.2 software and relatively quantitated using GAPDH as the internal control. The primers for all genes tested, including internal control GAPDH were synthesized by Gene Link: GAPDH: 5′-GTGGAGATTGTTGCCATCAACG-3′(sense), 5′-CAGTGGATGCAGGGATGATGTTCTG-3′ (antisense); TGF- [symbol] 1: 5′-GTGGTATACTGAGACACCTTGG-3′ (sense), 5′-CCTTAGTTTGGA CAGGATCTGG-3′ (antisense); IL-10: 5′-CTCTTACTGACTGGCATGAGG-3′ (sense), 5′-CCTTGTAGACACCTTGGTCTTGGAG-3′ (antisense); Foxp3: 5′-CAGCTGCCTACAGTGC C CCTAG-3′ (sense), 5′-CATTTG CCAGCAGTGGGTAG-3′ (antisense); arginase 1: 5′-CAGAGTATGACGTGAGAGACCAC-3′ (sense), 5′-CAGCTTGTCTACTTCAGTCATGGA G-3′ (antisense); iNOS: 5′-GAGATTGGAGTTCGAGACTTCTGTG-3′ (sense), 5′-TGGC TAGTGCTTCAGACTTC-3′ (antisense). For quantitative real-time PCR, 2 [symbol] l of cDNA reversely transcribed from total RNA was amplified by real-time quantitative PCR with 1× Syber green universal PCR Mastermix (Bio-Rad, Richmond, Calif.). Each sample was analyzed in duplicate with the IQ-Cycler (BioRad) and the normalized signal level was calculated based on the ratio to the respective GAPDH housekeeping signal.

### Results and Discussion

MSCs were tested to determine whether they can suppress the activated T cell immune response and induce Treg development. Gr-1⁺/CD11b⁺Fr. II cells and Gr-1⁻/CD11b⁺ Fr. II cells, derived from bone marrow and spleen of large MCA26 tumor-bearing BALB/c mice and sorted as described in Example 1, were irradiated (200 rad). Influenza hemaglutinin (HA)-specific CD4⁺ T cell receptor (TCR) transgenic T splenocytes from transgenic BALB/c mice were labeled with CFSE. The MSCs were then each co-cultured with the CFSE-labeled splenocytes in the presence of HA antigens, either HA peptide (CD4 HA peptide (¹¹⁰SFERFEIFPKE¹²⁰) or CD8 HA peptide (⁵³³IYSTVASSL⁵⁴¹)) or irradiated HA expression tumor cells. After 72 hr, cell division and CD25 (IL-2Rα) expression of HA-specific T cells were analyzed by flow cytometry. Viable cells were isolated using lympholyde to separate the dead and live cells and stained with anti-CD25-allophycocyanin (anti-CD25-APC) and anti-CD4-phycoerythrin (anti-CD4-PE) or isotype matched control antibodies (eBioscience). The threshold values used to gate the dot-plots on CD4⁺ cells were set using the isotype control antibodies.

The basic principles of this flow cytometry experiment are as follows. The Fr. II cells were irradiated to activate the cells. HA antigen was present in the co-culture to activate the T cells and generate a T cell response, which includes the expression of the CD25 cell surface receptor. A greater signal due to APC then indicates a greater T cell response. CFSE is a small molecule conjugate. It becomes fluorescent only after entering a cell and having acetyl groups cleaved by intracellular esterases. For conjugation, the CFSE reacts with free amines within the cell. Since this conjugation is indiscriminate, cell death may occur, prompting selection of only viable cells for the experiment after the 72 hr incubation period. The CFSE-conjugated material is divided among proliferating daughter cells. Therefore, the signal due to CFSE will become diluted as the T cells proliferate. A CFSE signal similar to the signal determined prior to co-incubation of cells would indicate that those T cells have not undergone significant cell division. The flow cytometry experiments have been gated, using the PE signal, to those that express CD4 in order to detect only T cells, and thus only the T cell response, and not the Fr. II cells, cellular debris, etc.

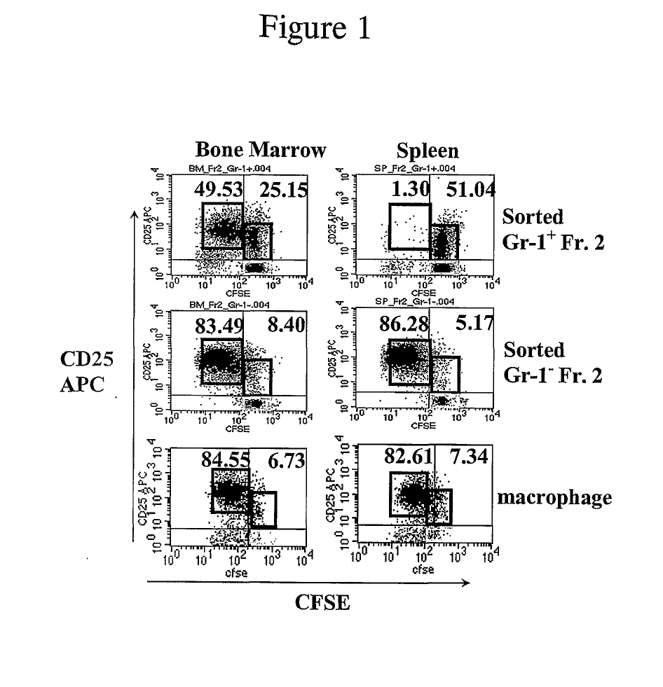

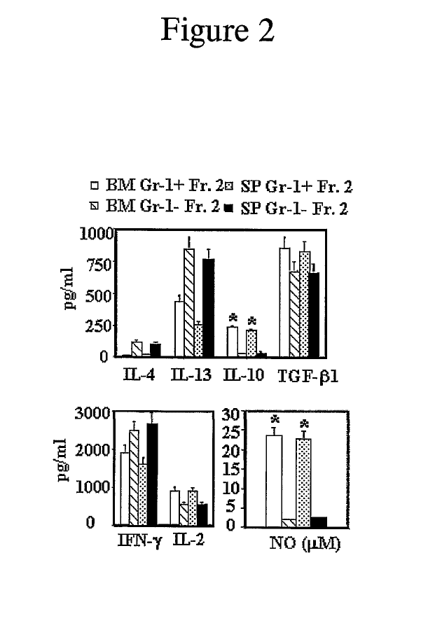

Gr-1⁺/CD11b⁺ Fr. II cells significantly inhibited the proliferation of CD4⁺ T cells whereas the Gr-1⁻/CD11b⁺ Fr. II and non-MSC macrophage cells did not (49% vs. 83% from BM, 1.3% vs. 86% from spleen; FIG. 1). A population of non-proliferating CD4⁺ cells that expressed a lower level of CD25 was observed in the co-culture with Gr-1⁺/CD11b⁺ Fr. II (25% from BM and 51% from spleen) while a very low percentage of CD4⁺/CD25⁺ non-dividing T cells was seen in the co-culture with control Gr-1⁻/CD11b⁺ Fr. II cells (8.4% from BM and 5.17% from spleen). These data reveal that a population of non-dividing CD4⁺ T cells that express the T cell activation marker CD25 are induced in the presence of Gr-1⁺ Fr. II MSCs, but not the control Gr-1⁻ Fr.2 cells. Consistent with these results, the evaluation of cytokine profiles and nitric oxide (NO) production in the supernatant showed significantly higher levels of IL-10 and NO and substantially higher levels of TGF-β and IL-2 in the co-culture with Gr-1⁺ Fr. 2 MSCs (FIG. 2). In contrast, higher levels of IFN-γ, IL-4, and IL-13 were detected in the supernatant of the co-culture with Gr-1⁻ Fr. 2 cells.

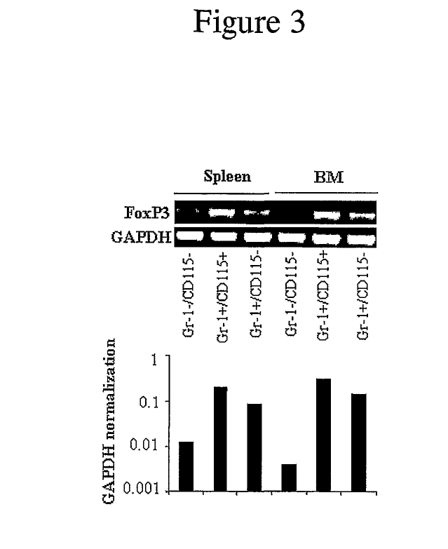

Antigen-specific T cell response after MSC stimulation was further characterized. To determine which specific cell population of Percoll Fr. II can induce Treg development, sorted irradiated Gr-1⁻/CD11b⁺/CD115⁻, Gr-1⁻/CD11b⁻/CD115⁻, Gr-1⁺/CD11b⁺/CD115⁻, and Gr-1⁺/CD11b⁺/CD115⁺ MSC cells (each at 97% purity) were co-cultured with HA-specific CD4⁺ TCR-transgenic T splenocytes for six days (single stimulation cycle only). Expression of Foxp3 as determined by reverse transriptase polymerase chain reaction (RT-PCR) and real-time RT-PCR was significantly induced by Gr-1⁺/CD11b⁺/CD115⁺ MSC whereas no significant Foxp3 expression was detected in the co-culture with Gr-1⁻/CD11b⁺/CD115⁻ or Gr-1⁻/CD11b⁻/CD115⁻ cells (FIG. 3). A substantial, but low, level of Foxp3 was detected in T cells stimulated with Gr-1⁺/CD11b⁺/CD115⁻ MSC. Thus, the RT-PCR and real time RT-PCR profile indicates that activation of antigen-specific T cells in the presence of MSC may favor the development of these activated T cells into Treg cells.

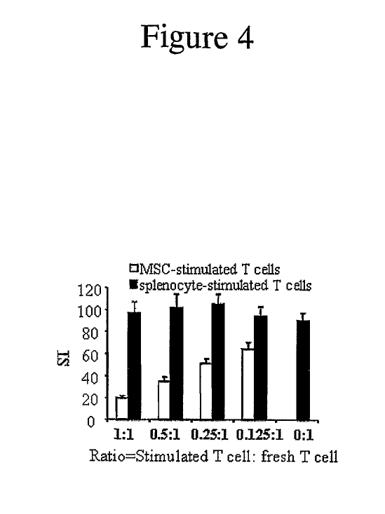

To confirm the suppressive function of T regulatory cells in the T cell plus MSC co-culture, Thy-1⁺ (CD90⁺) T cells were sorted from the co-cultures by fluorescence activated cell sorting (FACS). Thy-1⁺ was chosen to avoid activation of the T cells through binding of an antibody to CD4, etc. Thy-1⁺ T cells were sorted from the culture in the presence of irradiated HA MCA-26 cells with Gr-1⁺/CD11b⁺ MSCs or control splenocytes. The sorted T cells were co-cultured with splenocytes of naïve CD4⁺ HA-specific splenocytes (1×10⁵) in the presence of HA-peptide (1 μg/ml) at various cell ratios (1:1, 0.5:1. 0.25:1, 0.125:1) and tested for inhibitory activity in T cell proliferation assays. (The suppressive activity of MSC was assessed in a peptide-mediated proliferation assay of TCR transgenic T cells described above and previously (Li et al., Cancer Res. 2004, 64:1130-1139)). The sorted Thy-1⁺ plus MSC-co-cultured T cells significantly suppressed the proliferation of the fresh CD4⁺ HA-TCR T cells compared to CD4⁺ HA-TCR T cells co-incubated with control splenocytes (FIG. 4). Taken together, these data (the expression of Foxp3 and suppressive activity) provide strong evidence that Gr-1⁺/CD11b⁺/CD115⁺ MSC can induce the development of Treg cells. Treg development by MSC was further confirmed in vivo. These Treg cells can be depleted by CD25 antibody (see below).

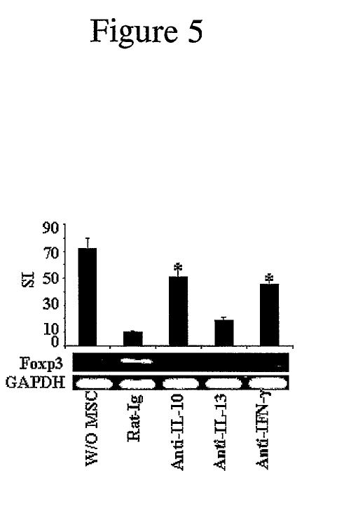

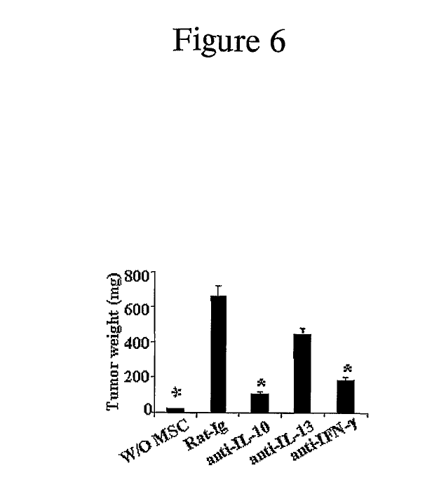

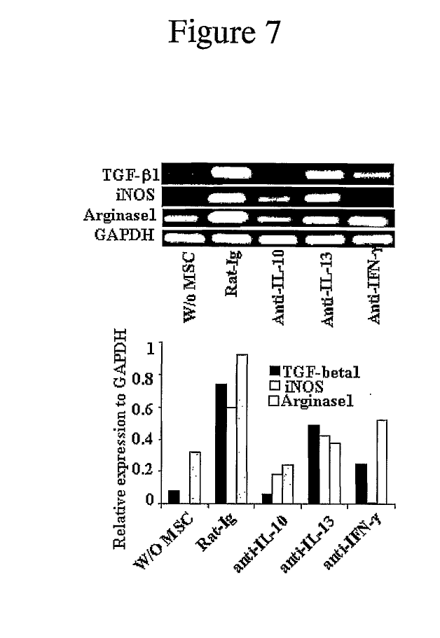

Since the high concentrations of IL-10, IL-13, and IFN-γ were detected in the supernatant of HA-specific CD4⁺ T cells co-cultured with MSCs and HA peptide (FIG. 2), whether these cytokines were necessary for the T cell anergy and Treg development induced by MSCs in vivo was investigated. MSC and T cell-co-adoptively transferred Thy1.1 tumor mice were simultaneously given intraperitoneal injections of control antibody (rat Ig), anti-IL-10, anti-IL-13, or anti-IFN-γ neutralizing antibodies. After 9 days, the adoptively transferred T cells were recovered by sorting for Thy1.2⁺ cells and their proliferative responses to HA peptide was evaluated and the level of Foxp3 gene expression was determined. Neither control antibody nor anti-IL-13 could reverse the hypo-proliferative response of sorted Thy1.2⁺ T cells (P=0.1093, ANOVA; FIG. 5). In contrast, treatment with anti-IL-10 or anti-IFN-γ antibodies (every three days at 150 μg/mouse/dose) significantly enhanced the proliferative response (P<0.01, ANOVA), which was accompanied by a significantly reduced level of Foxp3 (FIG. 5). In line with the above observation, the weight of dissected tumor tissue from the anti-IL-10 and anti-IFN-γ groups was significantly lower than that in mice from the control Ig-treated group (FIG. 6, P<0.01). Although the trend in the anti-IL-13 treatment group was toward some suppression of tumor growth, the observed decrease in tumor size did not reach statistical significance (P>0.05). The tumors were completely eradicated by the adoptively transferred HA-specific T cells in the mice that did not receive adoptive transfer of MSC (P<0.001). To determine the effect of anti-cytokine treatment on the tumor microenvironment, the expression levels of the TGF-β, iNOS (inducible nitric oxide synthase), and arginase 1 genes in the tumor tissue from animals in the various treatment groups were analyzed by RT-PCR. Anti-IL-10 treatment resulted in a 12-fold decrease in TGF-β gene expression and, to a lesser degree, iNOS (3-fold decrease) and arginase 1 (4-fold decrease) gene expression, when compared to treatment with the control antibody, rat Ig (where the intensity of amplified DNA bands was analyzed by IQ Mac 1.2 software and relative expression levels were compared to the internal control GAPDH, FIG. 7). IFN-γ is required for iNOS expression in the tumor as anti-IFN-γ treatment completely inhibited the expression of iNOS. TGF-β and arginase 1 mRNAs were detectable, however, at a lower level in the tumors from mice treated with anti-IFN-γ antibody when compared to rat Ig treatment. Substantial levels of TGF-β, iNOS, and arginase 1 gene expression were still detected in the tumor tissues from mice treated with anti-IL-13 antibodies. Taken together, the results suggest that IL-10 and IFN-γ are required for the suppression of anti-tumor responses and the development of Treg cells mediated by MSC in recipient tumor-bearing mice.

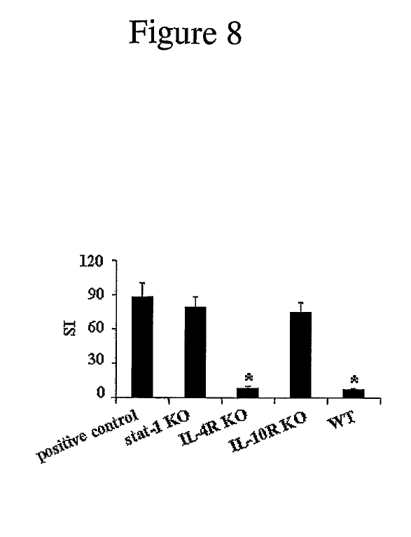

In addition, a comparable approach with mice deficient in signaling of Stat1 (Stat1^{-/-}), IL-4/IL-13 (IL-4Rα^{-/-}), or IL-10 (IL-10R^{-/-}) were used to confirm the role of IFN-γ, IL-13, and IL-10 in the suppression of anti-tumor responses mediated by MSCs. MCA26 and B16 tumor models were used in knockout mice with BALB/c and C57BL/6 backgrounds, respectively. The MSCs from wild-type or knockout tumor mice were co-adoptively transferred with T cells (HA-TCR in BALB/c and OVA-TCR in C57BL/6) into irradiated tumor (HA-MCA26 or OVA-B16)-bearing mice. Seven days later, the adoptively transferred T cells were recovered by FACS (Thy-1.2, BALB/c) or by T cell-enrichment column (C57BL/6). The proliferative response of recovered T cells to peptide stimulation was assessed. Consistent with the data from experiments using neutralizing antibodies, T cells recovered from mice that received MSCs deficient in Stat-1 (IFN-γ signaling) or IL-10R exhibited normal proliferative responses to peptide stimulation when compared to those recovered from the mice that did not receive MSCs (FIG. 8). T cells recovered from mice receiving wild-type or IL-4/IL-13 signaling deficient MSCs were hypo-proliferative in response to peptide stimulation. Moreover, the tumor mass of the mice that received IL-4Rα^{-/-} or wild-type MSCs was larger than that in mice that were injected with Stat1^{-/-} or IL-10R^{-/-} MSCs.

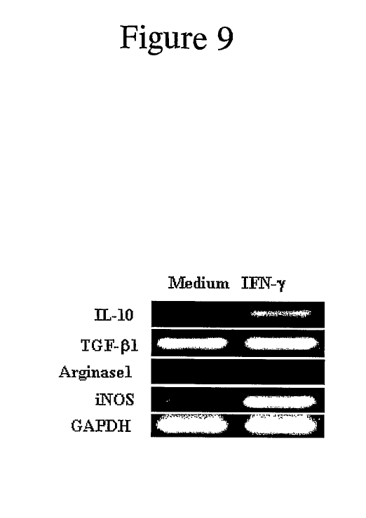

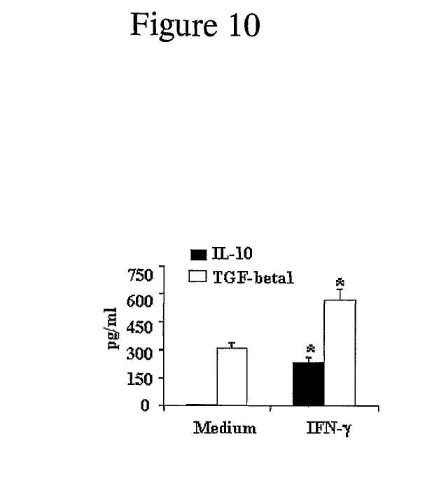

IL-10 and TGF-β have been shown to induce the development of Treg cells (Groux et al. Nature. 1997, 389:737-742; Wakkach et al. Immunity. 2003, 18:605-617; Seo et al. Immunology. 2001, 103:449-457; Fu et al. Am. J. Transplant. 2004, 4:1614-1627; Fantini et al. J. Immunol. 2004, 172:5149-5153; Chen et al. J. Exp. Med. 2003, 198:1875-1886.) Significant levels of IL-10 and TGF-β, along with IFN-γ, were detected in the supernatants of the co-culture of MSCs and CD4 HA TCR transgenic splenocytes (FIG. 1). Hence it was further hypothesized that MSC can secrete IL-10 and TGF-β in response to the stimulation of IFN-γ secreted by activated T cells. To test this hypothesis, Gr-1⁺/CD115⁺ MSCs were sorted, by FACS, from Percoll Fr. 2 derived from mice with large tumor burdens and cultured in the presence (100 ng/ml) or absence of IFN-γ. After stimulation for 24 hrs, the expression of IL-10, TGF-β, arginase 1, and iNOS genes and the secretion of IL-10 and TGF-β were assessed. TGF-β was expressed by sorted MSCs even in the absence of stimulation by IFN-γ (FIG. 9). The expression of IL-10 was not detectable without stimulation, but was induced in the presence of IFN-γ. Consistent with previous findings using bulk Percoll Fr. 2 cells, the expression of iNOS by sorted Gr-1⁺CD115⁺MSCs was significantly induced upon stimulation with IFN-γ. No arginase 1 mRNA was detected in the absence or presence of IFN-γ. In agreement with the RT-PCR results, significant levels of IL-10 and TGF-β were secreted by sorted MSC upon stimulation with IFN-γ (FIG. 10). Interestingly, the secretion of TGF-β by sorted MSCs was further enhanced in the presence of IFN-γ. The fact that there was no significant difference in TGF-β gene expression upon stimulation by IFN-γ when measured by RT-PCR is probably due to saturated amplification of primers (FIG. 9). No IL-2, IL-4, or IL-13 was detected in the culture supernatants in the absence or presence of IFN-γ. The data suggest that, upon stimulation by IFN-γ secreted from activated T cells, Gr-1⁺CD115⁺MSCs can secrete IL-10, TGF-β, and nitric oxide.

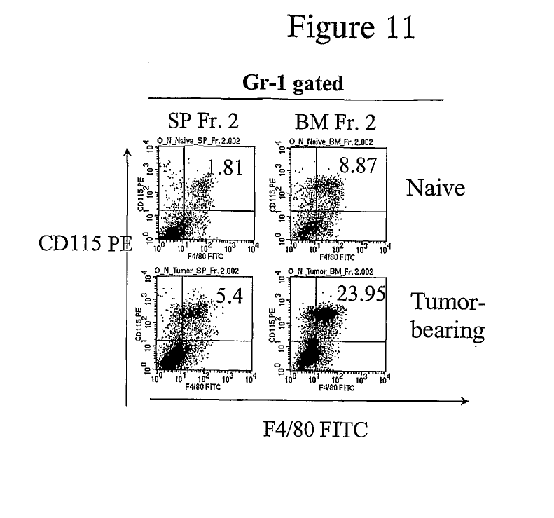

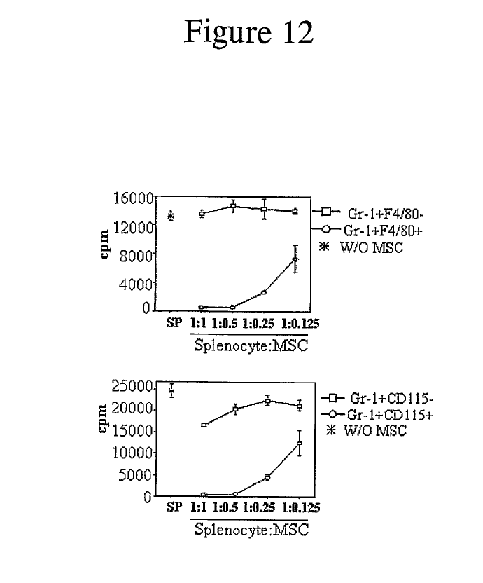

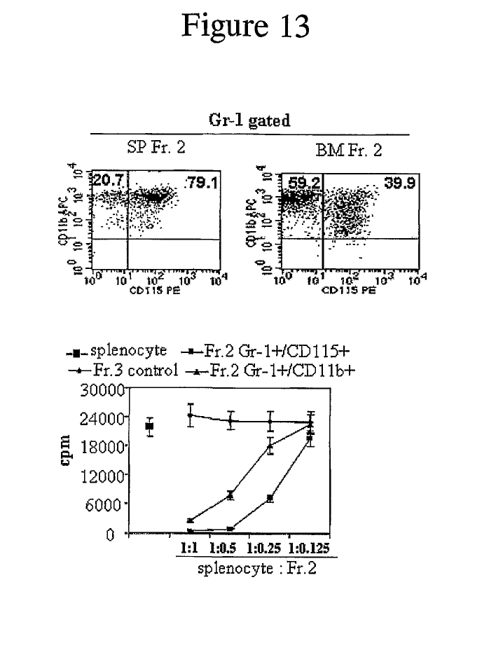

Cells sorted using F4/80⁺ were also studied. Percoll Fr. 2 cells derived from bone marrow (BM) and spleen of naïve or tumor-bearing mice were labeled with fluorochrome-conjugated antibodies. The Gr-1-gated flow cytometric profile (FIG. 11) showed a significantly increased percentage of Gr-1⁺/CD115⁺/F4/80⁺ cells in tumor-relative BM (23.95%) and spleen (5.4%) Fr. 2 compared with naive BM (8.87%) and spleen (1.81%) Fr. 2. and the absolute number of cells was even higher in the former. To determine whether the increased Gr-1⁺/CD115⁺/F4/80⁺ cells have suppressive function, tumor BM Percoll Fr. 2 cells were sorted into Gr-1⁺/F4/80⁺ vs. Gr-1⁺/F4/80⁻ or Gr-1⁺/CD115⁺ vs. Gr-1⁺/CD115⁻ populations for analysis of their suppressive activities in HA peptide-mediated proliferation assays. The strong suppressive effect of sorted Gr-1⁺/F4/80⁺ and Gr-1⁺/CD115⁺ cells, but not Gr-1⁺/F4/80⁻ or Gr-1⁺/CD115⁻ cells, was observed (FIG. 12). Based on the facts that 1) the majority of Gr-1⁺/CD115⁺ cells also expressed F4/80 and 2) CD115 is an earlier marker of myeloid progenitor cell than F4/80 (Anderson et al. Blood. 1999, 94:2310-2318), Gr-1 and CD115 were used to purify MSC from Percoll fraction 2. To address whether Gr-1 and CD115 are better markers for MSC than classical Gr-1 and CD11b, the percentage and suppressive function between the conventional MSC markers Gr-1⁺/CD11b⁺ and Gr-1⁺/CD115⁺ in Fr.2 cells were compared. All of the Gr-1⁺/CD115⁺ cells expressed CD11b makers. A stronger suppressive activity (˜2-fold increase) was observed in sorted Gr-1⁺/CD115⁺ cells when compared to sorted Gr-1⁺/CD11b⁺ cells (FIG. 13). Taken together, the results indicate Gr-1 and CD115 may be better markers to further enrich MSCs.

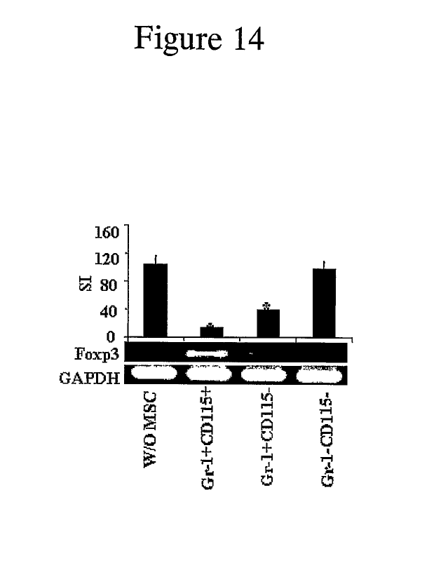

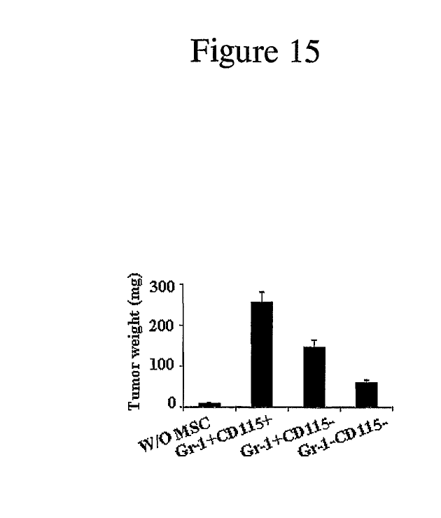

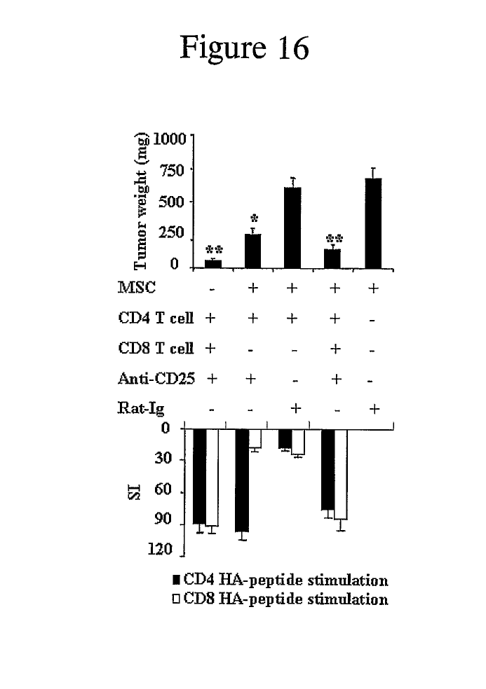

Whether antigen specific immune suppression in tumor-bearing mice was mediated through MSCs was further investigated. The sorted Gr-1⁺/CD115⁺ Fr. 2 MSCs, Gr-1⁺/CD115⁻ or Gr-1⁻/CD115⁻ Fr.2 cells (2.5×10⁶ cells/mouse) in conjunction with congenic Thy1.2⁺/CD4⁺/HA-TCR⁺ T cells (5×10⁶) were adoptively transferred into Thy1.1⁺ mice bearing HA-MCA26 tumors (5×5 mm²). One group only received T cell adoptive transfer but did not receive MSC as a negative control for Treg development. Before adoptive transfer, mice were irradiated to eradicate endogenous MSCs and T cells. Seven days later, Thy1.2⁺ T cells were sorted for the analysis of Foxp3 gene expression and proliferation assay. As shown in FIG. 14, a significantly higher level of Foxp3 expression was detected in the Gr-1⁺/CD115⁺ MSC group. In parallel with Foxp3 induction, T cells from Gr-1⁺/CD115⁺ group responded poorly to HA peptide stimulation whereas T cells from Gr-1⁻/CD115⁻ group proliferated vigorously. T cells from Gr-1⁺/CD115⁻ group proliferated upon stimulation by HA peptide, but at a significantly lower level when compared to Gr-1⁻/CD115⁻ group. The sorted Gr-1⁺ MSC (5×10⁶) and CD4⁺ HA-TCR⁺ T cell were co-adoptively transferred into HA-MCA 26 tumor bearing mice using the same strategy outlined above. More strikingly, the residual tumor weights were much lower in control splenocytes group or Gr-1⁻/CD115⁻ group (tumor mass: 0-25 mg), when compared Gr-1⁺/CD115⁺ group (tumor mass: 250-300 mg) (FIG. 15). To clarify whether tumor progression is ascribed to the effect of MSC-induced Treg, in vivo depletion of CD4⁺/CD25⁺ Treg by peritoneal injection of anti-CD25 antibody (PC-61, 100 μg/mouse) was performed. The depletion efficiency was confirmed by flow cytometry (>97%). The experimental group in which CD25⁺ T cells were depleted showed a significant reduction in tumor growth (FIG. 16). The adoptively transferred tumor-specific CD4⁺ or/and CD8⁺ T cells from the CD25 depletion groups (FIG. 16 lower panel), but not from the group without CD25 depletion, remained functional, indicating MSC-induced CD25⁺ Treg are involved in the suppression of anti-tumor responses. Taken together, the data suggest that adoptively transferred Gr-1⁺/CD115⁺ MSCs can render tumor (HA) specific T cells unresponsive to in vitro peptide stimulation, induce the development of CD25⁺ T cells that express Foxp3 and suppress anti-tumoral T-cell responses.

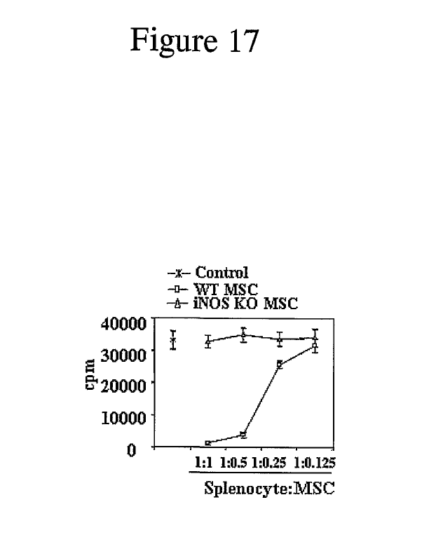

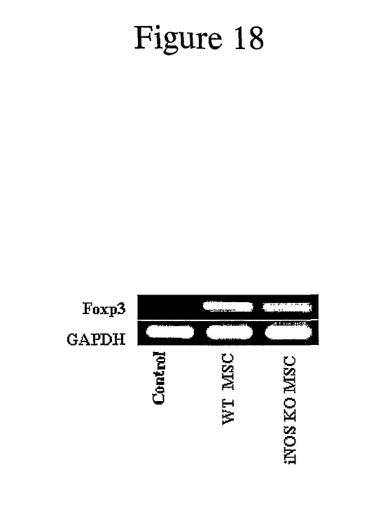

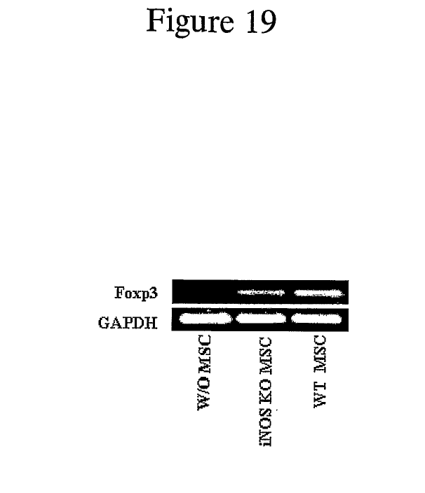

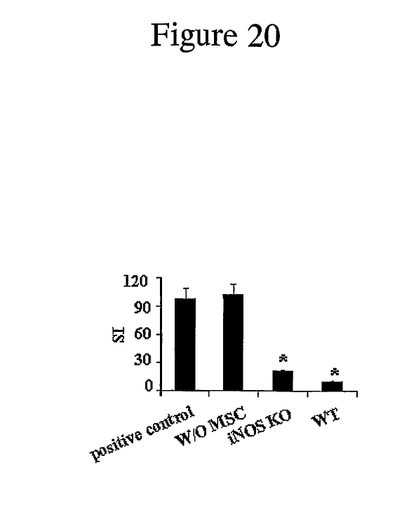

iNOS is required for MSC mediated immune suppression, but not required for Treg induction. Previous studies showed that IFN-γ-dependent NO production was required for the suppression of in vitro T-cell proliferation mediated by MSC. In this next experiment, whether NO production by MSCs is necessary for the development of Treg cells was studied. CD4 OVA TCR transgenic splenocytes were co-cultured with Percoll Fr. 2 Gr-1⁺ MSCs derived from wild-type or iNOS deficient tumor-bearing mice in the presence of irradiated OVA-B16 melanoma cells. Percoll Fr. 3 cells derived from wild-type tumor bearing mice were used as negative control. Six days later, cells were harvested and the expression of Foxp3 was analyzed by RT-PCR. In addition, the ability of iNOS deficient MSC to suppress T-cell proliferation was assessed. Consistent with previous findings, iNOS deficient MSC completely lacked suppressive activities (FIG. 17). However, a significant level of Foxp3 expression was still detectable in the co-culture with iNOS deficient MSC (FIG. 18). To further verify whether the expression of iNOS by MSC is required for the development of Treg cells in vivo, MSCs were isolated from iNOS deficient tumor-bearing mice and injected via the tail vein into irradiated OVA-B16 tumor-bearing mice that also received CD4 OVA TCR transgenic T cells. At day seven after adoptive transfer, OVA TCR transgenic T cells in the spleen were recovered. The proliferative response and Foxp3 expression of recovered T cells were assessed. A similar level of Foxp3 expression by T cells recovered from mice that received iNOS deficient MSCs was detected when compared to those from mice that received wild-type MSCs and the T cells still exhibited a hypo-proliferative response to peptide stimulation (FIGS. 19 and 20). The data suggest that the production of NO by MSCs is not required for the induction of Foxp3 expression and that both wild-type and iNOs deficient MSCs can induce the hypo-proliferation of T cells isolated from tumor-bearing mice.

### Example 3

### Myeloid Derived Suppressor Cells Mediated Immune Suppression to Prevent Type I Diabetes and Treg Development

### Mice

CD4-HA-TCR-Tg mice (BALB/c, H-2^{d}) expressed the 14.3.d HA-specific TCR, which recognizes the influenza hemagglutinin (HA, 110-120) epitope of A/PR/8/34 influenza virus in association with I-E^{d}. Ins-HA/RAG^{-/-} mice (B10.D2.H-2d) expressed the HA protein from the same virus in pancreatic β cells under the control of the rat insulin promoter. Mice were housed in pathogen-free conditions and were used according to the guideline of the Institutional Animal Care Committee at Mount Sinai School of Medicine.

### Tumor Model

The MCA26 tumor cell line is a BALB/c-derived, chemically induced colon carcinoma line with low immunogenicity. To generate the tumor model of metastatic colon cancer, MCA26 tumor cells (7×10⁴) were innoculated in the liver by intrahepatic implantation of cells as described previously (Huang et al., 2006. Cancer Res., 66: 1123, which is incorporated by reference in its entirety).

### Peptide and Antibodies

CD4 HA peptide (¹¹⁰SFERFEIFPKE¹²⁰) and CD4 ovalbumin peptide (³²³ISQAVHAAHAEINEAGR³³⁹) were purchased from Washington Biotechnology, Inc. (Baltimore, Md.). Biotin-conjugated anti-mouse ly-6G (Gr-1), Biotin-conjugated anti-mouse Thy1.1, anti-mouse CD4-FITC, anti-mouse CD25-APC, anti-mouse FoxP3-PE and isotype-matched monoclonal antibodies were purchased from eBioscience (San Diego, Calif.).

### Isolation of MSC

Mice with tumor sizes greater than 10×10 mm² were sacrificed and their spleen, tibias, and femurs were harvested. After lysis of RBC, bone marrow cells and splenocytes were fractionated by centrifugation on a Percoll (Amersham Biosciences, Uppsala, Sweden) density gradient as described (Shapiro et al., Diabetes, 2001, 358 Suppl:S21). Cell bands between 40% and 50% were labeled as fraction 1, between 50% and 60% as fraction 2, and between 60% and 70% as fraction 3. Cells were collected respectively from the Fraction 2 (Fr.2 cells) and Fraction 3 (Fr.3 cells). Then, Gr-1⁺ CD115⁺ MSC were sorted from Fr.2 cells by flow cytometry. Very stringent gating conditions were used (FACSVantage with FACSDiVa) and the purity of the MSC were up to 98%.

### Diabetes Model and Treatment

Thy1.2 congenic CD4-HA-TCR transgenic T cells were enriched by T-cell enrichment columns according to the manufacturer's instructions (R&D Systems) for adoptive transfer through tail vein injection (2×10⁷ or 1×10⁵ per mouse). 24 hours later 5×10⁶ sorted Gr-1⁺ CD115⁺ MSC from tumor bearing mice, with HA (5 μg/mouse) or with control peptide (OVA peptide), or control Fr.3 cells with the HA peptide were adoptively transferred into the recipient mice twice, at two day intervals. Some mice were injected with PBS as a mock injection control or MSC. The glucose levels of the mice were monitored with blood glucose meter (Bayer) daily to follow the onset of diabetes. Mice were considered diabetic when glycemia was >200 mg/dl after two consecutive measurements.

### Histopathological Analysis

Some pancreata were fixed in a 10% solution of buffered formalin embedded in paraffin, and then sections were cut in stair-wise (7 μm per section). Staining was done using the Mayer hematoxylin-eosin (H&E) technique. For each organ, ten sections were analyzed. Staining for intracellular insulin was done with polyclonal rabbit anti-insulin (Santa Cruz Biotechnologies Santa Cruz, Calif.) and revealed with a horseradish peroxidase (HRP)-goat-anti-rabbit conjugate (Southern Biotechnologies, Birmingham, Ala.). Some pancreata were frozen in −80° C. and then sections were cut in stair-wise (8 μm per section). Staining for CD4 T cells in islet was done with monoclonal anti-CD4 (eBioscience) and revealed with a horseradish peroxidase (HRP)-goat-anti-mouse conjugate (Southern Biotechnologies, Birmingham, Ala.).

### Proliferation Assay

T cells from non-diabetics/diabetics mice were recovered by MACS (Miltenyi Biotec, Inc) using biotinylated anti-Thy-1.2 antibody and their functional activities were assessed. Thy1.2 T cells (1×10⁵) with irradiated (2,500 rad) naive splenic cells (5×10⁴) as APC were cocultured with or without HA peptide (5 Ag/mL) in 96-well microplates. [³H] thymidine was added during the last 8 hours of 72-hour culture.

### Cytokine Detection by ELISA

The culture supernatants were harvested from above proliferation assay before [³H] thymidine was added during the last 8 hours of 72-hour culture. Cytokine ELISAs were done on the culture supernatants using the mouse IFN-γ, IL-10 and TGF-β ELISA kits (R&D Systems) according to the manufacturer's instuctions.

### Suppression Assay

The suppressive activity of CD25⁺ T cells, column-enriched from non-diabetic mice, was assessed in a peptide-mediated proliferation assay of TCR transgenic T cells as described previously (Huang et al., 2006, Cancer Res., 66:1123). Briefly, column-enriched Thy1.2 T cells (1×10⁵) from TCR transgenic mice with irradiated (2,500 rad) naive splenic cells (5×10⁴) as APC were cocultured with HA peptide (5 μg/mL) in the presence of serial dilutions of CD25⁺ T cells in 96-well microplates. [³H] thymidine was added during the last 8 hours of 72-hour culture.

### Quantitative Real-Time PCR

Target cells were homogenized in TRIzol reagent (Invitrogen) and total RNA was extracted according to the manufacturer's instructions. A reverse transcription-PCR (RT-PCR) procedure was used to determine relative quantities of mRNA (One-step RT-PCR kit; Qiagen). Twenty-eight PCR cycles were used for all of the analyses. The intensity of each amplified DNA bands was further analyzed by IQ Mac version 1.2 software and relatively quantitated using glyceraldehyde-3-phosphate dehydrogenase (GAPDH) as the internal control. The primers for all genes tested, including internal control GAPDH, were synthesized by Gene Link: GAPDH 5′-GTGGAGATTGTTGCCATCAACG-3′ (sense) and 5′-CAGTGGATGCAGGGATGATGTTCTG-3′ (antisense), Foxp3 5′-CAGCTGCCTACAGTGCCCCTAG-3′ (sense) and 5′-CATTTGCCAGCAGTGGGTAG-3′ (antisense), For quantitative real-time PCR, cDNA (2 AL) reverse transcribed from total RNA was amplified by real-time quantitative PCR with 1_SYBR Green Universal PCR Mastermix (Bio-Rad, Richmond, Calif.). Each sample was analyzed in duplicate with the IQ-Cycler (Bio-Rad) and the normalized signal level was calculated based on the ratio to the respective GAPDH housekeeping signal.

### Results and Discussion

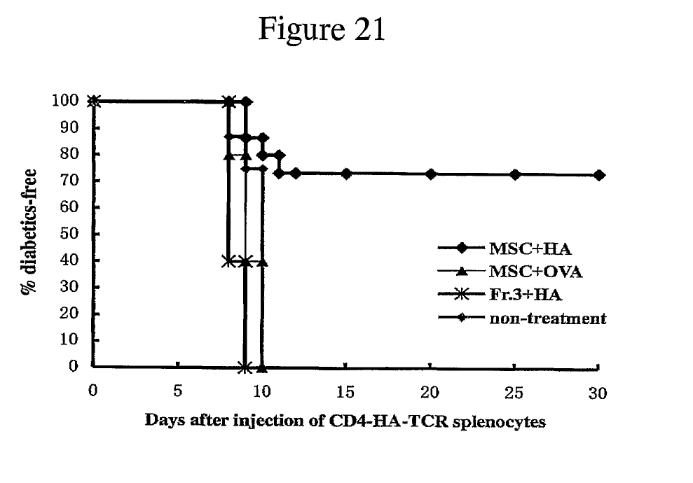

Studies of autoimmune response against natural autoantigens are hindered by the polyclonal nature of autoreactive T cells and the lack of traceable markers for in vivo studies. To study the suppressive effect of MSC on auto-antigen specific T cells in vivo, a diabetic model employing the adoptive transfer of T cells from CD4-HA-TCR-Tg mice into Ins-HA/RAG^{-/-} mice has been used. Ins-HA/RAG^{-/-} mice develop diabetes within 7-10 days after T cell adoptive transfer (FIG. 21).

Whether MSC could prevent diabetes induced by activated CD4-HA-TCR T cells in the Ins-HA/RAG^{-/-} mice, in which the HA antigen is under the control of the insulin promoter, was tested. The purified T cells from CD4-HA-TCR-Tg mice, at 1×10⁵/mouse, were transferred into the Ins-HA/RAG^{-/-} mic. 24 h later the Gr-1⁺CD115⁺ MSC plus HA or with OVA control peptide, or control Fr. 3 cells with the HA peptide were adoptively transferred into the recipient mice twice at 5×10⁶/mouse. The glucose levels of the mice were measured daily to follow the onset of diabetes. Interestingly, all of the mice that received PBS mock injection, MSC with control peptide, or Fr.3 cells with HA peptide developed diabetes in 7-10 days, Only adoptive transfer of MSC with specific HA peptides can significantly suppress the autoreactive T-cell immune response against the islet cells and prevent the onset of diabetes in recipient mice (FIG. 21, P<0.005). The percentage of diabetes-free mice in the MSC with HA peptides group is around 75%, which indicates that MSC can prevent diabetes induced by activated CD4-HA-TCR T cells in the Ins-HA/RAG^{-/-} mice.

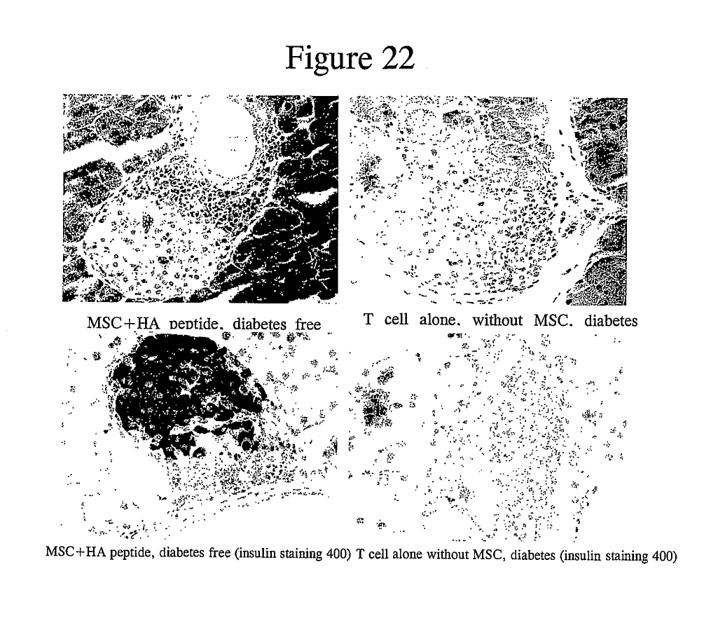

The degree and severity of insulitis in the various treated groups by the H&E staining was investigated. MSC with HA peptide treated mice had significantly reduced insulitis, as shown by a higher frequency of peri-islet insulitis and non-infiltrated islets compared to control mice (MSC with control OVA peptide or T cell transfer alone). Control mice developed massive intra-islet infiltration and lack of insulin production in most of the islets (FIG. 22, upper panel). Few MSC with HA peptide treated mice that developed diabetes also showed heavy pancreatic infiltration that was comparable to control diabetic mice (data not shown).

Insulin expression and islet integrity of treated mice were further determined by immunohistochemical analysis. Consistent with the blood glucose levels, no insulin expression or intact islets were detected in the pancreas taken from diabetic mice that were received with T cell transferred alone (FIG. 22 lower panel). In contrast, both insulin expression and intact islets were detected in non-diabetic mice that were treated MSC+HA peptide (FIG. 22, Lower left panel).

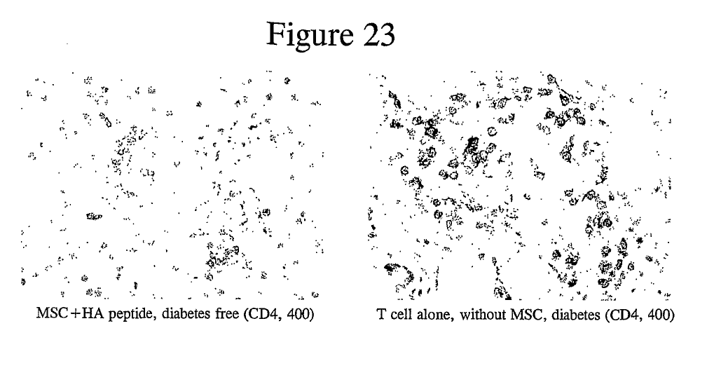

To determine the CD4 T cell infiltration in islets, CD4 expression in islets of treated mice was determined by immunohistochemical analysis. The results showed that the severity of infiltration in the diabetic-free mice treated with MSC+HA peptide was much more reduced than that in the other diabetic mice (FIG. 23). This suggests that MSC may inhibit the CD4 T cells infiltration into the β-islets which express HA peptide.

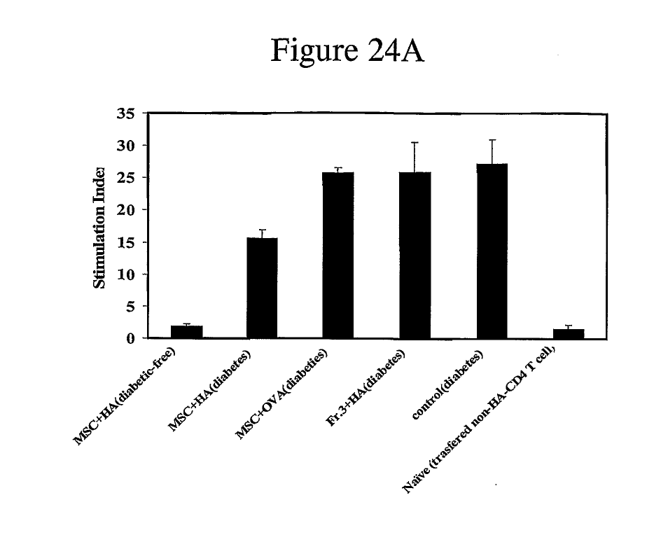

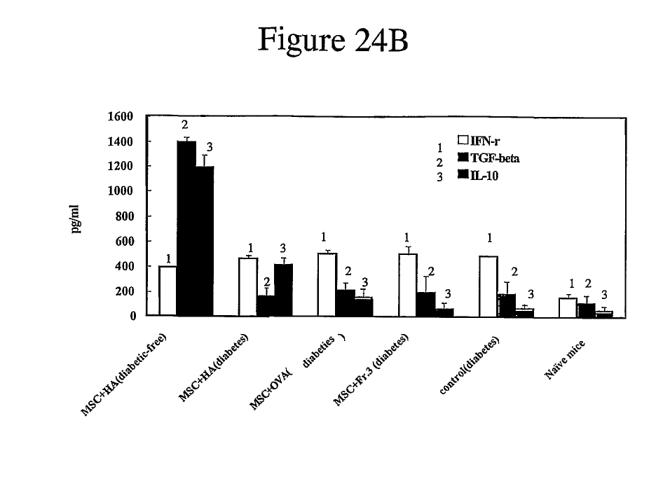

The effect of MSC on autoreactive CD4-HA-TCR T cells was analyzed. As shown in FIG. 24, column-enriched T cells, which were recovered from diabetic mice (MSC+OVA, Fr.3+OVA or no treatment group) proliferated well upon stimulation with HA peptide. Interestingly, while T cells recovered from diabetic mice (n=5) which have received transfer of MSC+HA still have some proliferation in the presence of HA and irradiated naïve splenocytes (as antigen presenting cells), however at a lower level. Those T cells recovered from non-diabetic mice (n=14) that received the transfer of MSC+HA did not proliferate significantly, suggesting that a state of non-responsiveness is induced.

To determine whether the inhibitory cytokines were secreted in the culture supernatants from the above T cell proliferation assay. The IFN-γ, IL-10 and TGF-β were measured. The results indicate that significantly higher levels of IL-10 and TGF-β were secreted in MSC+HA treated group compared to the other diabetic mice groups (FIG. 24B, P<0.001). However, there was no difference in the IFN-γ level between diabetes-free group and diabetes group (P>0.05), suggesting that inhibitory cytokines are secreted from the anergic T cell induced by MSC in the diabetic free mice.

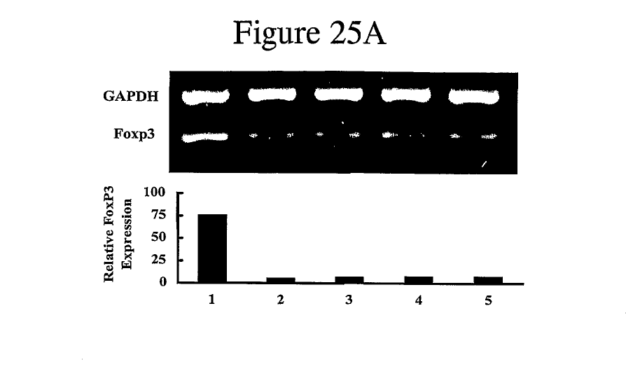

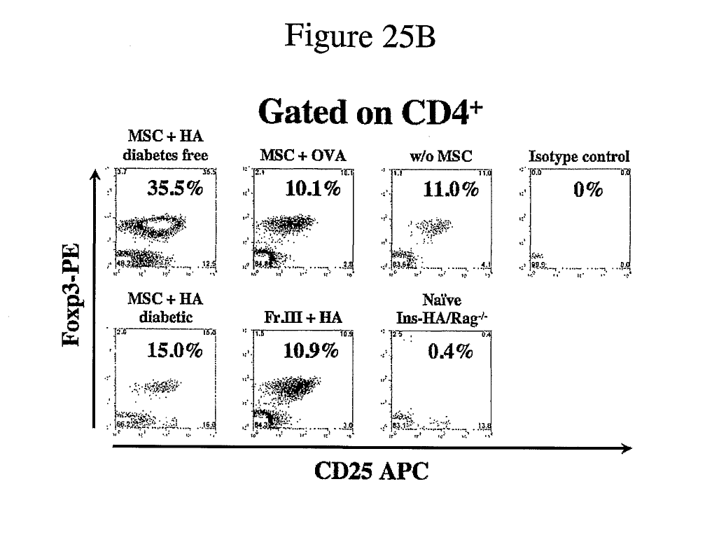

Since anergic T cells secrete significant amounts of TGFb and IL-10. Whether Tregs were induced upon transfer of MSC+HA peptide was determined. Foxp3, the transcriptional factor involved in Treg development and function, gene and protein expressions were assessed by real-time PCR and intracellular staining, respectively. As shown in FIG. 25A, a significantly higher level of Foxp3 gene expression was detected in T cells recovered from non-diabetic mice that received transfer of MSC and HA peptide (Lane 1) when compared to those recovered from diabetic mice that received transfer of MSC with HA peptide (Lane 2) or control OVA peptide (Lane 3) or Fr.3 with HA peptide (lane 4) or CD4-HA-TCR T cells alone, without MSC or Fr.3 cells (Lane 5). Consistent with the real-time PCR results, a significantly higher percentage (35.5%) of Foxp3⁺CD4⁺CD25⁺ T cells was observed in non-diabetic mice that received transfer of MSC and HA when compared to diabetic mice that received no treatment or transfer of MSC+OVA or control Fr.3 cells+HA (10.1% and 11.0%; FIG. 25B). Interestingly, a lower, but substantial, percentage (15.0%) of Foxp3⁺CD4⁺CD25⁺ T cells was detected in diabetic mice that received transfer of MSC and HA.

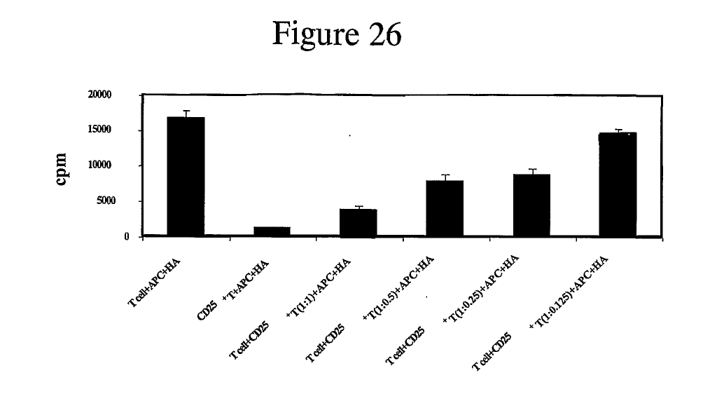

To determine whether these MSC-induced Treg in vivo can have immune suppressive activity and inhibit the activated T cell immune response, suppression assays were performed. CD25⁺T cells were isolated from non-diabetics mice (with MSC+HA peptide transferred) were co-cultured with T cells isolated from naïve CD4-HA-TCR transgenic mice and irradiated naive splenic cells as APC in the presence of HA peptide (5 μg/mL). Serial ration of CD25⁺T cells vs. Thy1 enriched CD4-HA-TCR T cells were tested. The results (FIG. 26) showed that CD25⁺T cells from non-diabetic mice significantly inhibit antigen specific T cell proliferation in a dose-dependent manner.

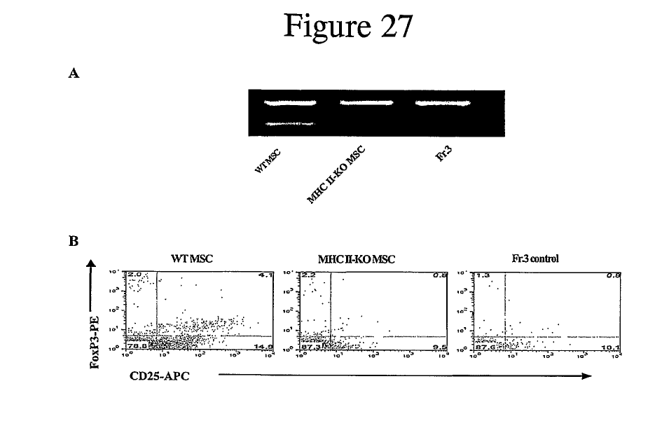

Whether the mechanism of MSC-mediated Treg induction requires direct antigen presentation was investigated. The MSC isolated from MHC class II KO mice were tested. The results indicate that MSC from MHC ClassII KO mice can not efficiently induce Foxp3 positive Treg cell as compared with MSC isolated from wild-type mice, which were confirmed by the real time RT-PCR and FOXP3 intra-cellular staining as shown in FIG. 27, A and B.

Since anti-CD40 can reverse T cell tolerance and prevent Treg induction, whether the expression of CD40 on MSC is required for Treg development and tolerance induced by MSC was investigated. Similar results have found that CD40 is required for Treg induction. The results indicate that MHC class II and CD40 expression on MSC is required for MSC mediated immune suppression and Treg induction. These results support the hypothesis that MSC can play a critical role in controlling autoimmune T cell response for immune tolerance and Treg induction.

### Example 4

### Identification of Cytokines Required for MSC Accumulation in Tumor-Bearing Animals

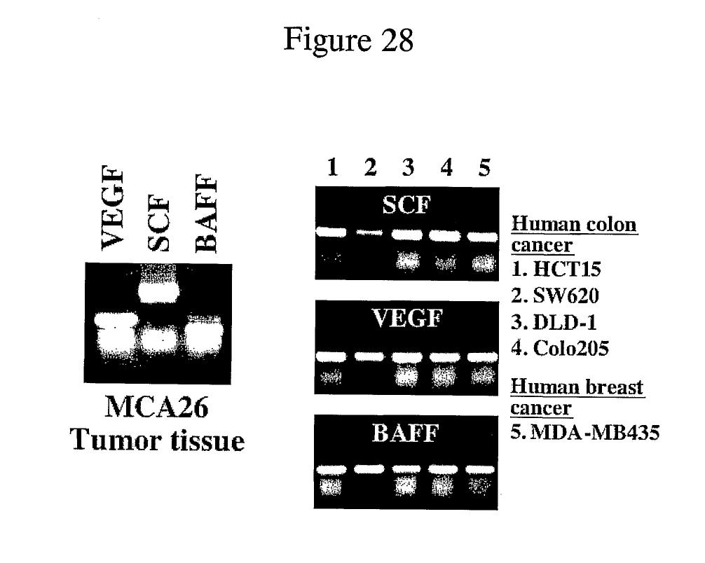

Cytokines were identified that are involved in MSC accumulation in tumor-bearing animals. The results of microarray analysis of various human and mouse cell lines indicate that stem-cell-factor (SCF) may be required for MSC accumulation in vivo. Also, significant levels of M-CSF (assayed through the use of a M-CSF dependent cell line), and GM-CSF (6.4 pg/ml) were detected in 48-hr culture supernatants of MCA26 colon carcinoma cells. To identify additional candidate tumor factors, gene expression profile analysis of MCA26 tumor tissues were performed using GEAray Q Series Mouse Common Cytokines Gene Array (SuperArray), which contains 96 common mouse cytokine genes. Several candidate genes were identified and their expressions were confirmed using RT-PCR in multiple murine and human tumor cell lines from multiple tissue origins (FIG. 28). M-CSF, GM-CSF, stem cell factor (SCF, alternately known as c-kit ligand, mast cell growth factor, and steel-factor, among others), BAFF (B-cell activating factor, also known as BLyS, TALL-1, THANK, zTNF4, or TNFSF13B), and VEGF (VEGF-A) are secreted and present in the tumor cells.

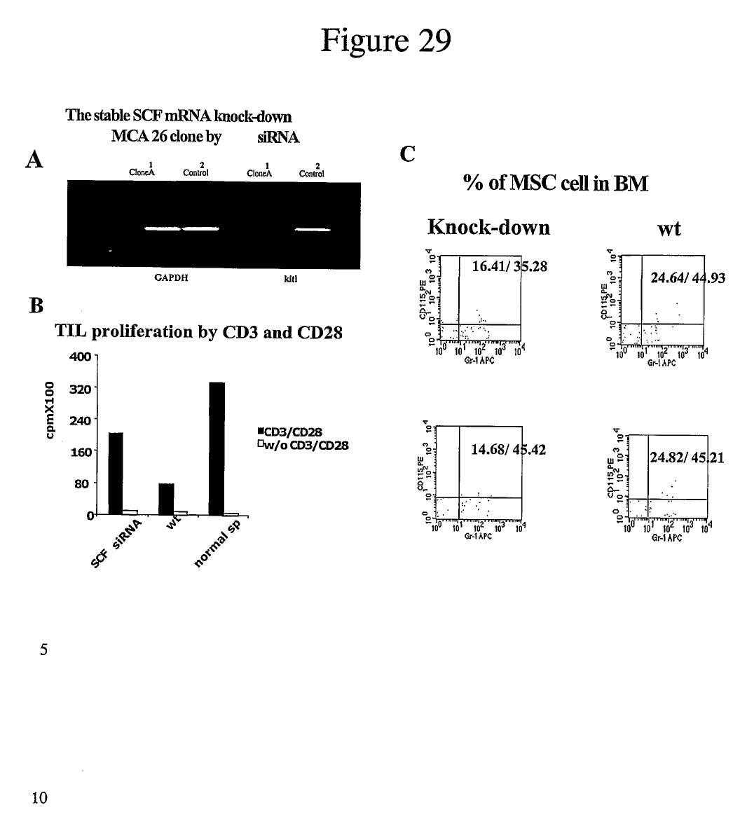

As shown by RT-PCR (clone A, FIG. 29A), a stable SCF knockdown MCA-26 cell line was established using siRNA specific for SCF using a plasmid from Ambion following manufacturer's instructions. Bone marrow Percoll Fraction 2 cells from mice bearing SCF knockdown MCA-26 tumors vs. normal (wt) MCA-26 tumors (two mice per group) were stained with anti-Gr-1-APC and anti-CD115-PE or isotype control antibodies and analyzed by flow cytometry. This experiment showed that mice bearing SCF knockdown MCA-26 tumors had significantly less Gr-1⁺/CD11b⁺/CD115⁺ MSC (14-16%) in bone marrow when compared to those bearing mock-transfected control MCA-26 tumors (24.7%) (FIG. 29C).

Tumor-infiltrating lymphocytes were isolated from control or SCF knockdown MCA-26 tumor tissues. The anti-CD3/anti-CD28 mediated proliferative responses of the T cells were assessed in a standard [³H]-thymidine incorporation assay. The T cells isolated from the SCF knockdown tumor tissue exhibited a higher proliferative response to anti-CD3/anti-CD28 stimulation when compared to those from control tumor tissue (FIG. 29B).

### Example 5

### In Vitro Generation of MSC from Primary Cultures of Hematopoietic Stem Cells (HSC) in the Presence of SCF

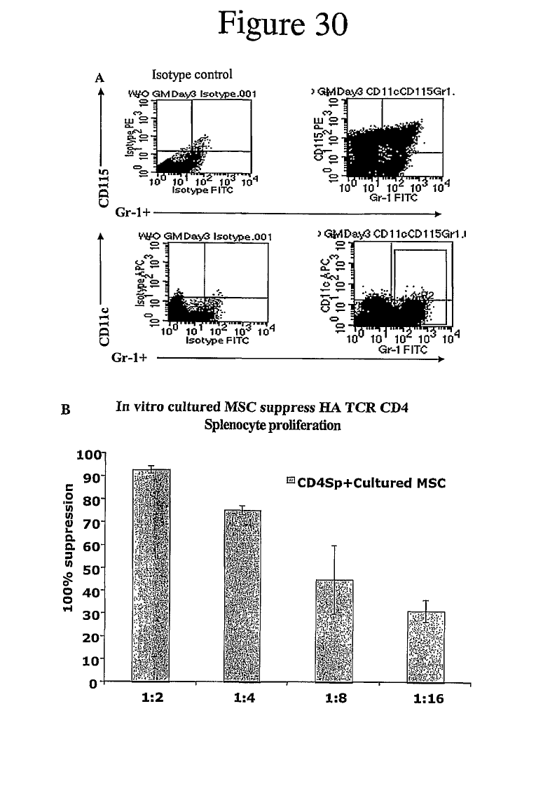

Since SCF is required for MSC accumulation in tumor-bearing mice, MSCs were further tested to determine whether they can be generated by in vitro culture. The primary HSC isolated from normal mouse bone marrow (as described in Example 1 where further sorting can be achieved using Sac and c-kit) were cultured in the presence of SCF. Three days later, cells were harvested and stained with anti-Gr-1-FITC+anti-CD115-PE+anti-CD11c-APC or isotype control antibodies. The primary cultures of HSC differentiated into Gr-1⁺/CD11b⁺/CD115⁺ MSC (FIG. 30A, upper right panel). The in vitro derived Gr-1⁺/CD11b⁺/CD115⁺ MSCs (Percoll Fr. II) were co-cultured with naïve CD4⁺ HA-specific splenocytes (1×10⁵) in the presence of HA-peptide (1 μg/ml) at various ratios of MSCs (1:2, 1:4, 1:8, 1:16). These MSCs suppressed the proliferation of CD4⁺ HA TCR transgenic T cells (FIG. 30B).

### Example 6

### MSC-Mediated Suppression of Allo-Immune Response and GVHD

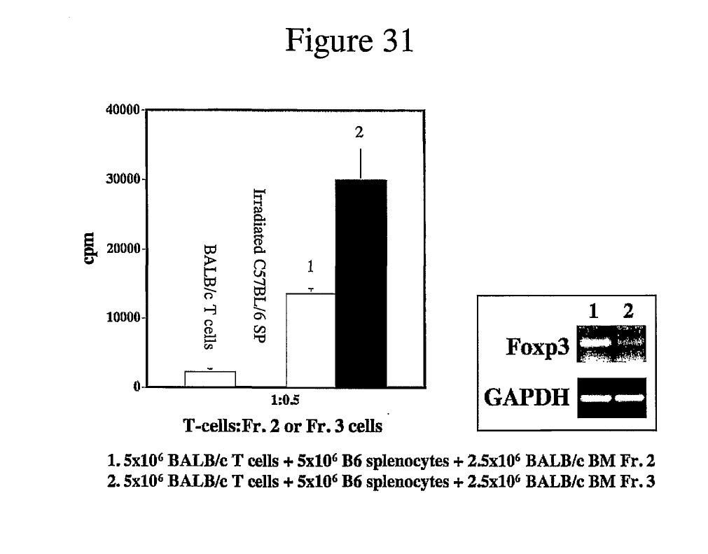

The suppressive effect (suppression of T-cell proliferation and Treg induction) of MSC on allogeneic mixed lymphocyte reaction (MLR) was assessed. Purified T cells from BALB/c mice (responders) were co-cultured with irradiated C57BL6 splenocytes (stimulators) in the presence of MSC (Fr. 2) or control Percoll Fr. 3 cells. The results indicate that MSC, but not control Percoll Fr. 3 cells, can not only suppress the proliferation of MLR but also induce Foxp3 gene expression (FIG. 31).

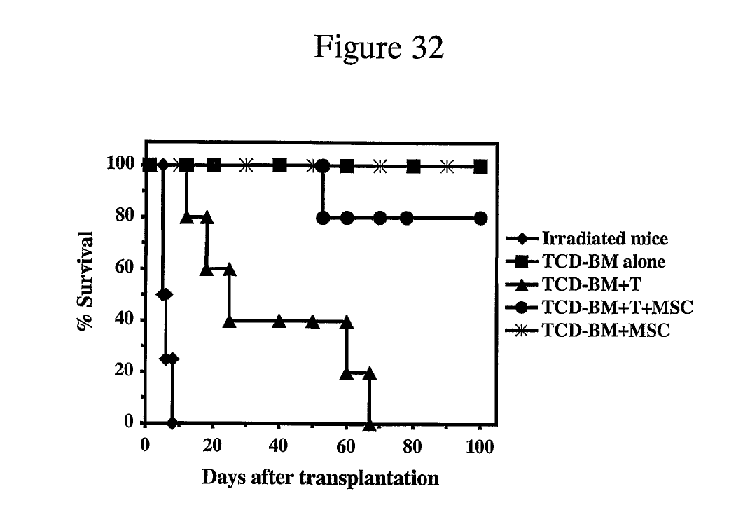

Whether MSC can suppress the allo-specific T-cell response in a GVHD (graft vs. host disease) model was tested. BALB/c mice (6-8 weeks old) were lethally irradiated with 10 Gy and 4 hours later transplanted with T-cell depleted bone marrow cells (BM) (C57BL/6) alone, T cell depleted-BM (C57BL/6) and purified splenic T cells (C57BL/6), T cell depleted-BM (C57BL/6)+purified MSC(C57BL/6), or T cell depleted-BM (C57BL/6)+purified splenic T cells (C57BL/6)+purified MSC(C57BL/6). The survival of treated mice in each group was followed (FIG. 32). All irradiated mice without any BM transfer or with adoptive transfer of BM+T cells died within one week and 70 days respectively. Interestingly, 80% of the mice that received BM+T cells+MSC did not develop GVHD and survived for more than 100 days. H-2K^{b+} cells were detected in those mice, suggesting the establishment of chimerism.

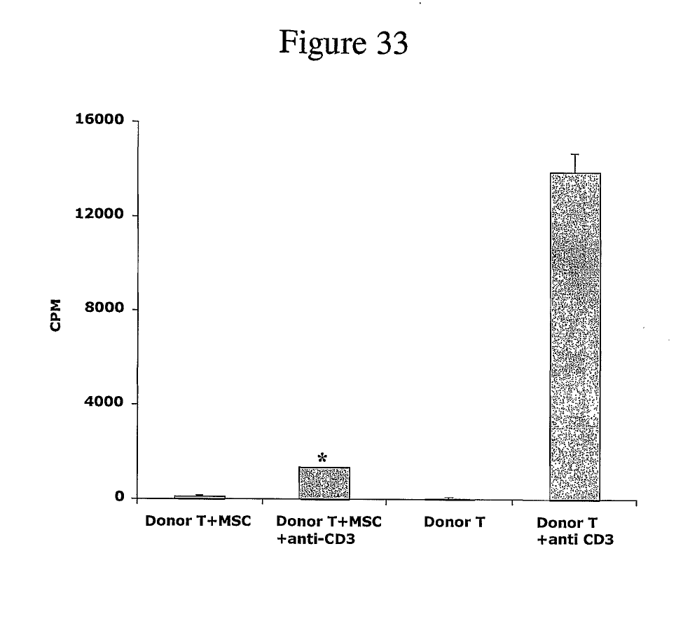

To determine the mechanisms by which MSC suppress the allo T cell response in this GVHD model, donor T cells were recovered by sorting from mice that received BM+T cells (before the mice succumbed to death) or BM+T cells+MSC and the proliferative response mediated by anti-CD3 was assessed. As shown in FIG. 33, T cells isolated from irradiated hosts that received BM+T cells proliferated significantly in the presence of anti-CD3 (the 4^{th} column from the left) whereas T cells isolated from mice that received BM+T cells+MSC did not (the 2^{nd} column). These results suggest that a state of anergy is induced in the donator C57BL6 T cells in recipient mice that received BM+T cells+MSC.

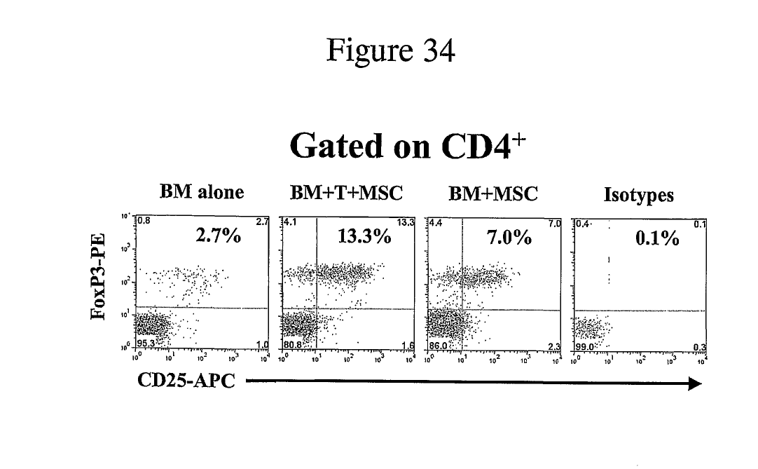

The development of CD4⁺CD25⁺Foxp3⁺ T cells in treated mice was also analyzed. Splenocytes from treated mice were stained with anti-CD4-FITC+anti-CD25 APC+anti-Foxp3-PE or isotype control. The percentage of CD4⁺CD25⁺Foxp3⁺ T cells was analyzed by flow cytometry. Interestingly, a higher percentage of CD4⁺CD25⁺Foxp3⁺ T cells was found in mice that received BM+T cells+MSC when compared to those that received BM alone or BM+T cells (FIG. 34; 13.3% vs. 2.7% or 7.0%). The result suggests that a portion of donor T cells have become CD4⁺CD25⁺Foxp3⁺ T cells.

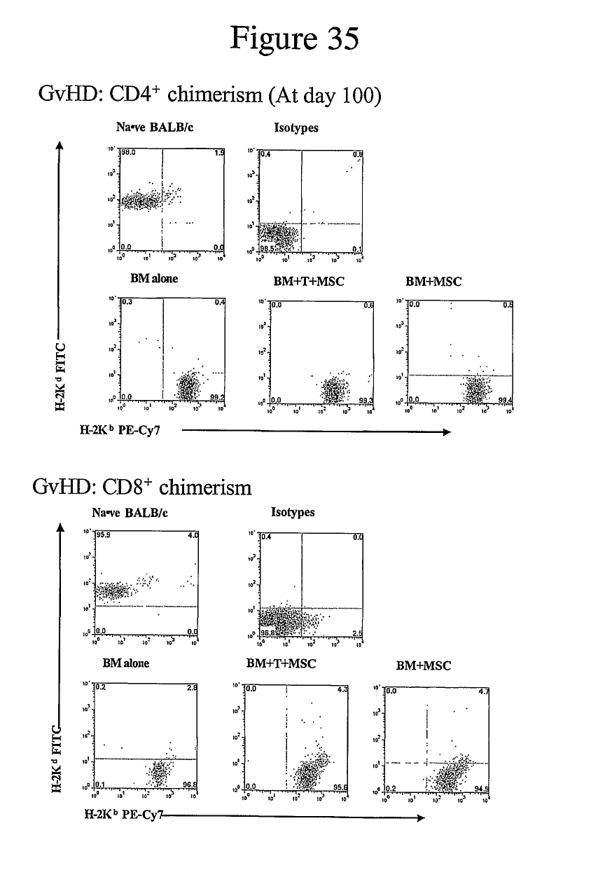

Whether the long-term survival mice after of allo-T cell and MSC transfer become the chimerism. The MHC class I haplotye of CD4 and CD8 T cell were analyzed. The recipient BABL/c (H-2 Kd) mice have completely developed the H-2 Kb CD4 (FIG. 35, top) and CD8 T cell (FIG. 35, bottom) after 100 day after long-term survival.

Taken together, our preliminary results suggest that MSC can suppress allo-T cell responses by the induction of T-cell anergy and development of CD4⁺CD25⁺Foxp3⁺ T cells in a GVHD model.

### Example 7

### MSCs Enhance the Eradication of Host T Cells and Induce the T Cell Tolerance after Co-Transfer with Donor T Cells

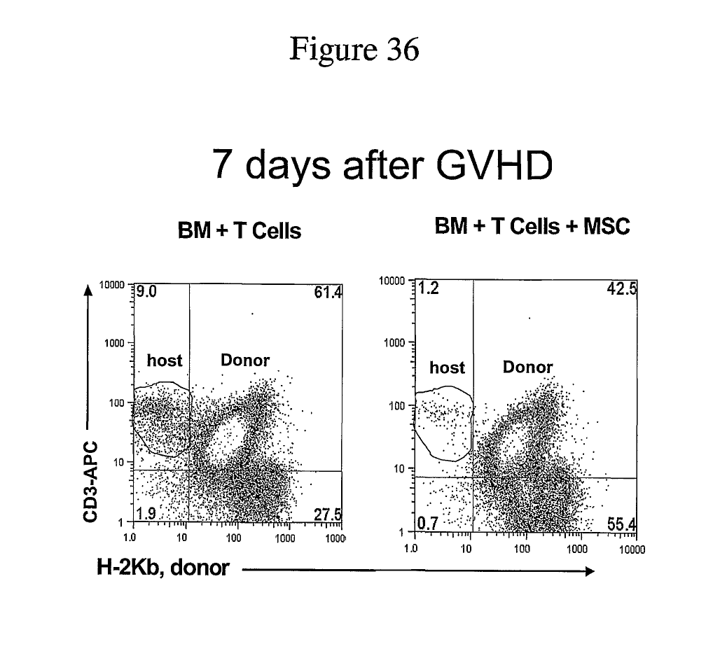

The congenic mouse (Thy1.1) system was used and the adoptively transferred donor T (Thy1.2) cells from recipient mice (Thy1.1) at day 7 of adoptive transfer were recovered by FACS (co-stained with anti-CD3 and donor specific anti-H-2K^{b} antibodies). Blood samples were collected from the recipient mice and co-stained with anti-CD3 and donor specific anti-H-2K^{b} antibodies. The mice receiving T-cell depleted BM and donor T cells or BM+T cells+Gr-1⁺/CD11b⁺ (can be Gr-1⁺/CD115⁺ or Gr-1⁺/F4/80⁺). MSCs have a significant number of H-2K^{b} positive leukocytes, indicating that chimerism has been established in the recipient mice. A significantly higher number of CD3 and donor H-2K^{b} positive leukocytes were detected in the mice receiving BM+T or BM+T+MSC. Host T cells were significantly less in the recipient mice that received MSCs. The co-transfer of T-cell depleted bone marrow cells with T cells and MSC facilitated the eradication host-derived T cells (CD3⁺/H-2 K^{b} negative) and the development of chimerism (see FIG. 36).

The proliferate response of donor T cells was further tested using anti-CD3 stimulation (FIG. 33). The sorted donor Thy1.2 T cell from congeneic Thy1.1 host were tested for ant-CD3 mediated T-cell proliferation. T cells (1×10⁵) from mice receiving BM+T cell alone or BM+T+MSC were stimulated with anti-CD3 antibody (1 μg/ml) for 72 hours. [³H]-Thymidine was added for the last 8 hours of co-culture. The T cells from mice that received bone marrow cells+T cells+MSC exhibited a significantly lower proliferate response, suggesting that T-cell anergy was induced by MSC. The T cells isolated from mice receiving only bone marrow cells and T cells still proliferated upon stimulation with anti-CD3.

### Example 8

### Reversion of Immune Tolerance by Modulation of Myeloid Derived Suppressor Cell Development in Advanced Malignancy

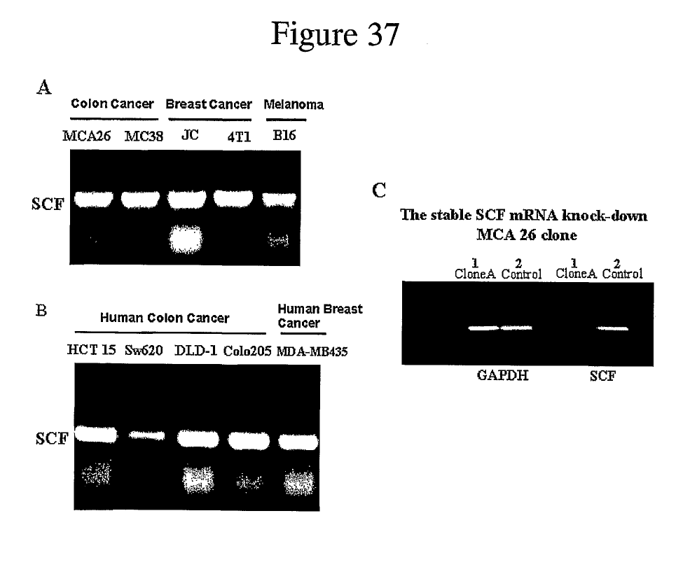

To identify candidate tumor factors that are involved in MDSC accumulation, a gene expression profile analysis of MCA26 tumor tissues using GEArray Q Series Mouse Common Cytokines Gene Array (SuperArray) was performed. Several candidate genes were identified, and one of the most highly expressed cytokines in tumor cell lines and tumor tissue was found to be SCF. The expression of SCF was further confirmed using RT-PCR in multiple murine and human tumor cell lines e.g. colon, breast, melanoma from multiple tissue origins (FIG. 37A, B).

Since SCF, also known as steel factor, mast cell growth factor, and c-kit ligand, plays an essential role in early and late stages of hematopoiesis, it was hypothesized that SCF secreted by tumor cells may regulate the accumulation of MDSC by simultaneously enhancing myelopoiesis and may attenuate monocyte/granulocyte/DC differentiation.

Whether SCF is involved in MDSC accumulation in tumor-bearing animals was investigated. A stable stem-cell-factor (SCF) knockdown MCA26 cell line using siRNA specific for SCF (FIG. 37C) was established. Mice bearing SCF knockdown MCA26 tumors had significantly fewer Gr-1⁺CD115⁺MSC (14-16%) when compared to those bearing mock-transfected control MCA26 tumors (24.7%). These results suggest that the ablation of tumor-derived SCF alone may have a significant impact on the accumulation of MSC. The effect of tumor size on the accumulation of MDSC (Gr-1⁺CD115⁺) in bone marrow fraction II (Fr. II) of SCF knockdown vs. parental tumor bearing mice was determined. The results indicate that mice bearing large (>10×10 mm²) or medium (7-10 mm²)-size SCF-knockdown tumors have fewer Gr-1⁺ cells when compared to those bearing parental tumors. A less significant difference was observed in mice with small tumors. More importantly, the bone marrow Fr. II cells from SCF knockdown tumor bearing mice exhibit less suppressive activity when compared to those from the parental tumor bearing mice (see FIG. 29). More interesting, the T cells isolated from the SCF knockdown tumor tissue exhibited a higher proliferative response to anti-CD3/anti-CD28 stimulation when compared to those from control tumor tissue, which indicates that there is significantly less T cell anergy from the SCF knockdown tumor tissue (FIG. 29). These results suggest that SCF secreted from tumor cells may play an important role in MDSC accumulation that, this in turn, may inhibit T cell activity.

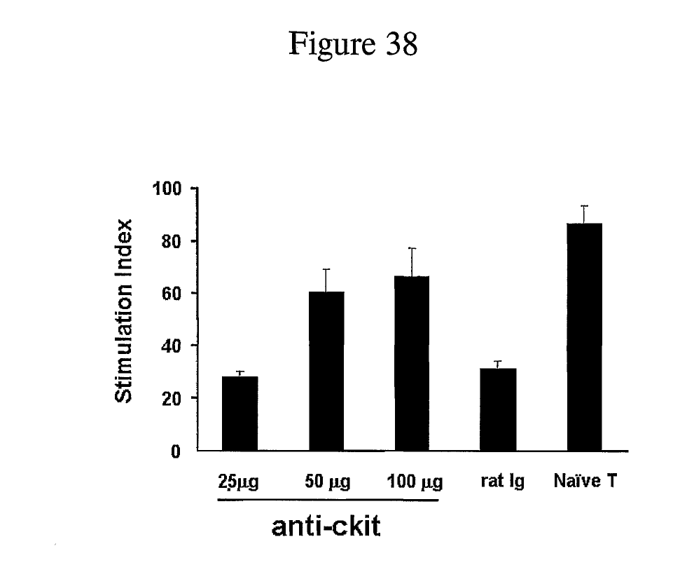

It was hypothesized that blocking SCF-signaling by anti-ckit (SCF receptor) antibody may reduce MDSC accumulation and prevent T cell anergy in mice with large tumor burdens. Mice bearing MCA26 tumors were injected with various doses of purified anti-c-kit antibodies every three days for a total of four doses. TILs were isolated from the anti-c-kit vs. control rat Ig (100 μg) treated animals and stimulated with anti-CD3 and anti-CD28. The results indicate that the low dose of 50 or 100 μg, but not the 25 μg, anti-ckit antibodies are sufficient to restore the T cell proliferation response as shown in FIG. 38.

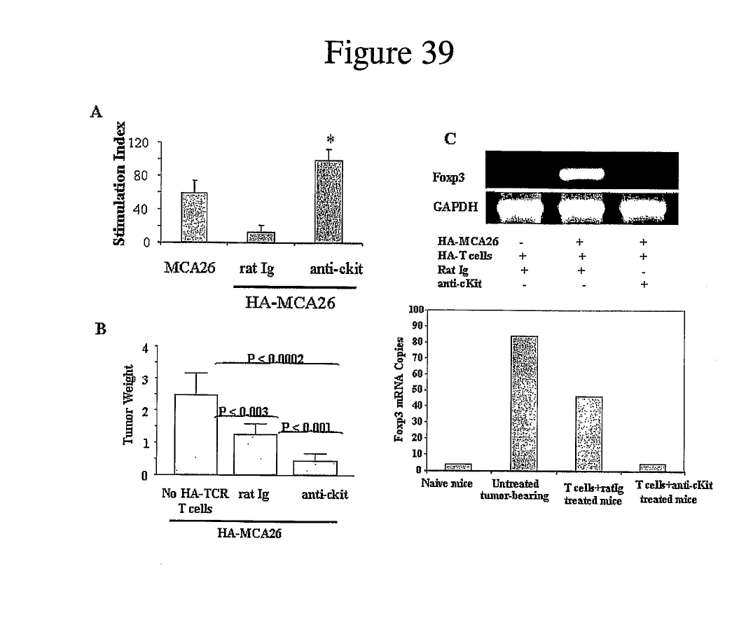

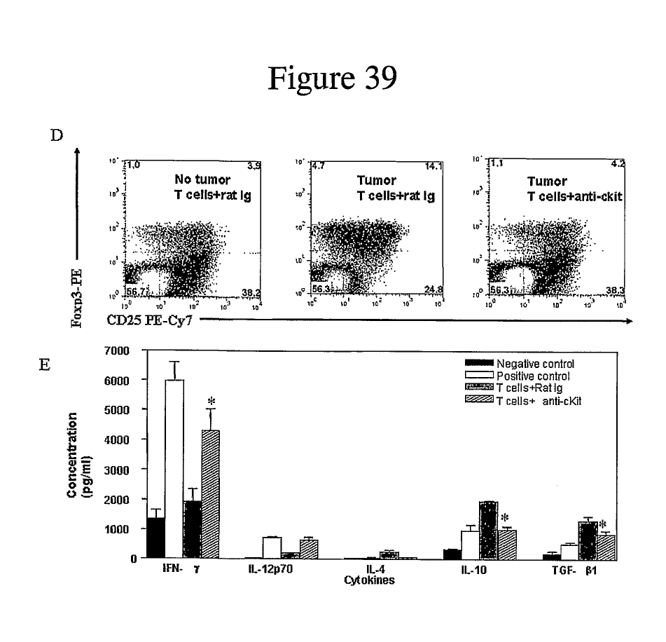

MDSC can mediate suppression of tumor-specific T cells responses in tumor-bearing animals. Whether blocking the accumulation of MDSC using anti-ckit antibodies can prevent tumor-specific T cell anergy in the HA-MCA26 tumor-bearing model ((Huang et al., 2006. Cancer Res., 66: 1123) was investigated. BALB/c mice were intrahepatically inoculated with HA-MCA26 tumor cells or control MCA26 tumor. At day 9, one group of mice was transferred with 5×10⁶ HA-TCR T cells and injected with control Ig, one group with HA-TCR T cells and anti-c-kit, one group with anti-c-kit, and the last group with rat Ig as a control. The immune response of adoptively-transferred tumor antigen-specific T cells (Thy1.2+ CD4 HA TCR transgenic T cells) in recipient Thy1.1⁺ HA-MCA26 or control MCA26 tumor-bearing mice treated with anti-ckit or control Ig (50 μg/mouse) every three days for four doses was assessed. The sorted Thy1.2⁺ CD4 HA TCR transgenic T cells isolated from rat Ig treated HA-MCA26 tumor bearing animals proliferated poorly in response to HA peptide stimulation. Interestingly, transferred TCR transgenic T cells recovered from anti-ckit treated HA-MCA26 tumor-bearing mice exhibited significantly higher proliferative responses to HA peptide when compared to those recovered from rat Ig-treated recipient mice. The proliferative response was even higher, although not significantly, when compared to that using cells isolated from MCA26 control (without HA antigen) tumor-bearing animals (FIG. 39A). The residual tumor tissue was dissected and weighed. The tumor weight from anti-ckit-treated animals was significantly lower than that of rat-Ig treated mice (FIG. 39B, P<0.001). More significantly, some of the mice that received anti-ckit treatment and transfer of TCR transgenic T cells became tumor-free (by pathological examination of the entire liver at the day of termination). The residual tumors from mice with anti-ckit treatment were pale in color and were less vascular when compared to those from the rat Ig treated tumor-bearing animals.

To determine the mechanism underlying the blockade of T cell tolerance by anti-ckit antibodies, tumor-specific (CD4 HA TCR transgenic) T cells were recovered from anti-ckit treated mice by cell sorting (Thy1.2⁺ cells). Foxp3 (a transcriptional factor specifically expressed by Treg) expression of the recovered tumor-specific T cells was analyzed by RT-PCR, real-time RT-PCR, and intracellular staining. As shown in FIG. 39C, tumor-specific T cells recovered from mice treated with control rat Ig expressed a high level of Foxp3, whereas those recovered from mice treated with anti-ckit expressed a significantly lower level of Foxp3. As shown in FIG. 39D, consistent with the results from RT-PCR analysis, a significantly lower percentage (4.2%) of Foxp3⁺ tumor-specific T cells was detected in mice receiving anti-ckit when compared to mice treated with control rat Ig (14.1%). Similar results have been reproduced from multiple animals from multiple experiments. Furthermore, upon in vitro stimulation with HA peptide, T cells recovered from mice treated with anti-ckit secreted higher levels of IFN-γ and IL-12, and lower levels of IL-10 and TGF-β when compared to those from mice treated with control rat Ig (FIG. 39E). The results demonstrate that anti-ckit can prevent T cell anergy and Treg development.

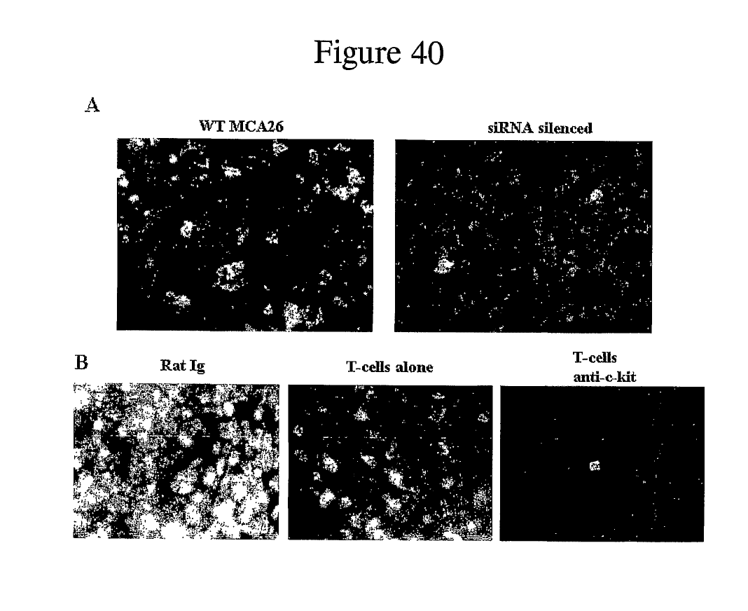

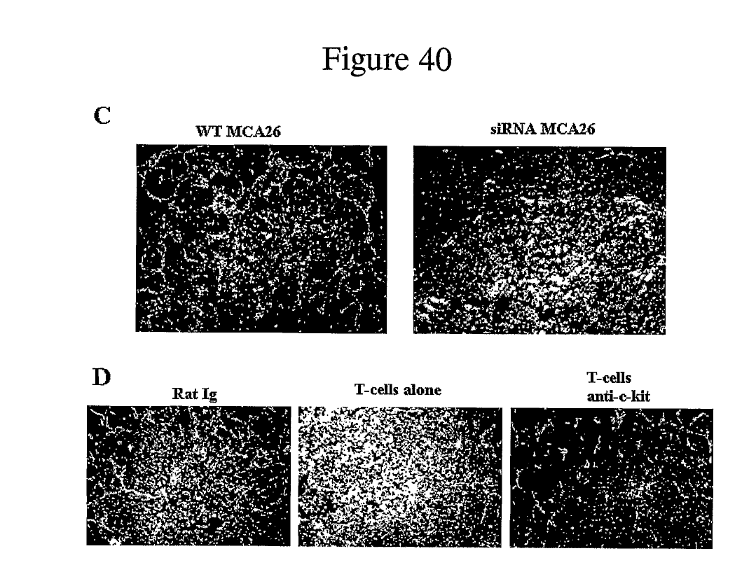

Whether blocking SCF signaling by anti-c-kit or with the SCF-siRNA silenced MCA 26 colon tumor tissue can significant reduce the MSC accumulation in situ by immunostaining was confirmed. As shown in FIG. 40A, B, the Gr-1+ MSC is significantly reduced in SCF-siRNA silenced tumor and anti-ckit treated tumor tissue as compared to wild type tumor or control rat Ig antibody treated tumor tissue. As previous report by Yang et al. (Cancer Cell 6: 409, 2004) has indicated that MDSC may enhance tumor angiogenesis, if MDSC accumulation can be prevented, this may also reduce angiogenesis, in theory, at the tumor site. There appears to be less blood vessels in the anti-ckit treated tumor tissue. This result was confirmed by immunostaining with anti-mouse CD31. The SCF-siRNA knockdown MCA26 tumor or the tumor tissue from mice which has been treated with T cell transfer and anti-ckit showed significantly reduced CD31 positive blood vessel formation when compared to WT tumors or to T cell transfer alone or in conjunction with control rat Ig injection tumor groups (FIG. 40C, D). The results indicate that adoptive transfer of tumor-specific (HA)-T cells has no effect on tumor angiogenesis, but the treatment of anti-c-kit antibody alone can significantly prevent the angiogenesis.

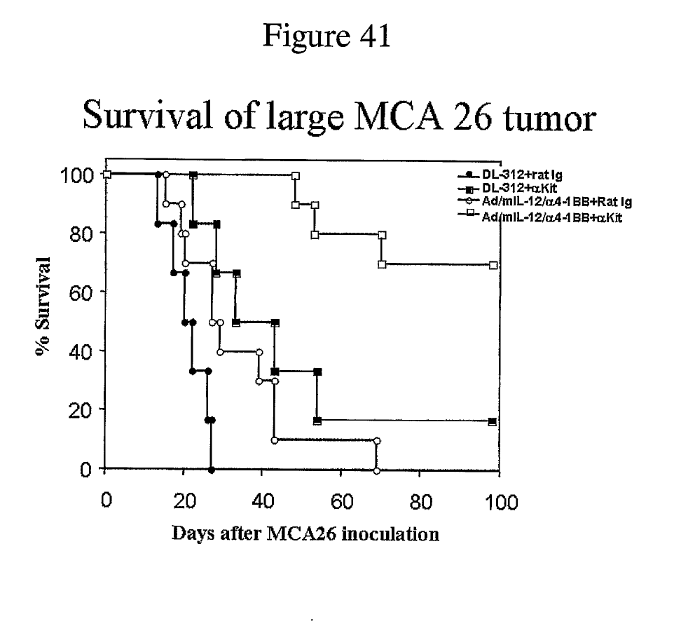

Activated immune therapy in large tumor-bearing animals is significantly hampered by immune tolerance has been demonstrated (Pan et al., 2002, Molecular Therapy, 6: 528-536). Since Tregs are involved in the down-regulation of anti-tumor responses, whether blockade of MDSC accumulation, Treg development, and T cell tolerance by anti-ckit could further enhance the therapeutic efficacy of Adv.mIL-12+4-1BB activation therapy was determined. Mice with large tumors (10×10 mm²) were divided into various treatment groups, Starting two days before initiation of the (IL-12+4-1BB) immune modulatory therapy, mice were injected intraperitoneally with anti-ckit or control rat Ig (50 μg) every three days for four doses. Anti-4-1BB or control Ig (100 μg) was injected intraperitoneally on days 1 and 3 after the injection of Adv.mIL-12 or control viral vector DL312. The long-term survival rate of treated mice was followed (FIG. 41). All mice treated with control vector DL312+ control Ig succumbed to death before day 30 after tumor implantation. The long-term survival rate of mice treated with anti-ckit+Adv.mIL-12+4-1BB activation is significantly higher than that of mice treated with Adv.mIL-12+4-1BB activation (P<0.0001). Adv.mIL-12+4-1BB activation alone (P=0.0165) or anti-ckit alone (P=0.0364) also improves the long-term survival of treated mice when compared with the control (DL312+Ig) treated group. The results demonstrate that treatment with anti-ckit can significantly improve the immune therapeutic efficacy of IL-12+4-1BB activation when treating large tumors. These results suggest that the prevention of MDSC accumulation may reduce MDSC mediated immune suppression and Treg induction, which may facilitate the successs of active immune cancer therapy.

### Example 9

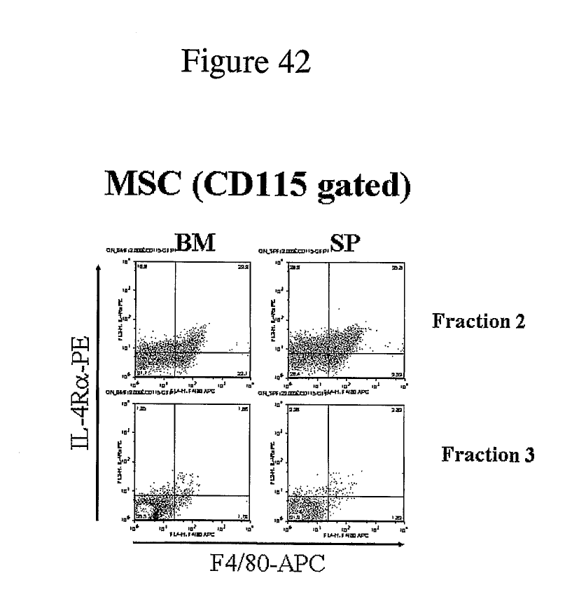

As shown in FIG. 42, some Fr. II CD115+/F4/80+ cells are also IL-4 receptor alpha positive. Some of the endogenous IL-13 can bind to the IL-4 receptor that may reduce the positive staining population. Around 30% MSC are positive. See Gallina et al., J. Clin. Invest., 2006, 116:2777-90.

The present invention is not to be limited in scope by the specific embodiments described herein. Indeed, various modifications of the invention in addition to those described herein will become apparent to those skilled in the art from the foregoing description and the accompanying figure(s). Such modifications are intended to fall within the scope of the appended claims.

Numerous references, including patents, patent applications, and various publications are cited and discussed throughout the specification. The citation and/or discussion of such references is provided merely to clarify the description of the present invention and is not an admission that any such reference is “prior art” to the present invention. All references cited and discussed in this specification are incorporated herein by reference in their entirety and to the same extent as if each reference was individually incorporated by reference.

1. A method of treating an autoimmune disease in an individual, which method comprises administering a therapeutically effective amount of myeloid suppressor cells (MSCs) to the individual, wherein the MSCs have a Gr-1⁺/CD11b⁺ phenotype.

2. The method according to claim 1 wherein the autoimmune disease is type I diabetes.

3. The method according to claim 1 wherein the MSCs are autologous.

4. The method according to claim 1 wherein the method further comprises administering an inhibitor of MSC terminal differentiation.

5. The method according to claim 4, wherein the inhibitor inhibits the activity of GM-CSF, M-CSF, or IL-3.

6. The method according to claim 1 wherein the method further comprises altering SHIP signaling.

7. The method according to claim 1 wherein the method further comprises increasing F4/80 expression.

8. The method according to claim 1, further comprising the step of administering one or more autoantigens.

9. The method according to claim 1 wherein the MSCs are genetically modified to express or overexpress one or more autoantigens.

10. The method according to claim 8, which method further comprises administering a cytokine.

11. The method according to claim 10 wherein the cytokine is IFN-γ, IL-10 or TGF-β.

12. The method according to claim 1 wherein method further comprises administering an immunosuppressive drug.

13. The method according to claim 12 wherein the immunosuppressive drug is cyclosporin, methotrexate, cyclophosphamide or tacrolimus.

14. The method according to claim 1, wherein the MSC phenotype further comprises CD115.

15. The method according to claim 1, wherein the MSC phenotype further comprises F4/80.

16. The method according to claim 1, wherein the phenotype includes at least one additional marker selected from the group consisting of CD31, c-kit, VEGF-receptor, and CD40.

17. The method according to claim 1, wherein the MSCs are genetically modified to overexpress Gr-1.

18. The method according to claim 1, wherein the MSCs are genetically modified to overexpress CD115.

19. The method according to claim 1, wherein the MSCs are genetically modified to overexpress F4/80.

20. A method of treating an alloimmune response in an individual, which method comprises administering a therapeutically effective amount of myeloid suppressor cells (MSCs) to the individual, wherein the MSCs have a Gr-1⁺/CD11b⁺ phenotype.

21. The method according to claim 20 wherein the alloimmune response is graft rejection.

22. The method according to claim 20 wherein the alloimmune response is graft-versus-host disease (GVHD).

23. The method according to claim 20 wherein the wherein MSCs are autologous.

24. The method according to claim 20 wherein the method further comprises administering an inhibitor of MSC terminal differentiation.

25. The method according to claim 24 wherein the inhibitor inhibits the activity of GMCSF, M-CSF, or IL-3.

26. The method according to claim 20 wherein the method further comprises altering SHIP signaling.

27. The method according to claim 20 wherein the method further comprises increasing F4/80 expression in MSCs.

28. The method according to claim 20, further comprising the step of administering one or more autoantigens.

29. The method according to claim 20 wherein the MSCs express one or more autoantigens.

30. The method according to claim 28 wherein method further comprises administering a cytokine.

31. The method according to claim 30 wherein the cytokine is IFN-γ, IL-10 or TGF-β.

32. The method according to claim 20 wherein method further comprises administering an immunosuppressive drug.

33. The method according to claim 32 wherein the immunosuppressive drug is selected from the group consisting of cyclosporin, methotrexate, cyclophosphamide, and tacrolimus.

34. The method according to claim 20, wherein the MSC phenotype further comprises CD115.

35. The method according to claim 20, wherein the MSC phenotype further comprises F4/80.

36. The method according to claim 20, wherein phenotype includes at least one additional marker selected from the group consisting of CD31, c-kit, VEGF-receptor, and CD40.

37. The method according to claim 20, wherein the MSCs are genetically modified to overexpress Gr-1.

38. The method according to claim 20, wherein the MSCs are genetically modified to overexpress CD115.

39. The method according to claim 20, wherein the MSCs are genetically modified to overexpress F4/80.

40. A method of producing myeloid suppressor cells (MSCs), which method comprises culturing primary hematopoietic stem cells (HSCs) in the presence of stem-cell factor (SCF) in an amount and for a time sufficient to allow HSCs to differentiate into MSCs, wherein the MSCs have a Gr-1⁺/CD11b⁺ phenotype.

41. The method according to claim 40, wherein the MSC phenotype further comprises CD115.

42. The method according to claim 40, wherein the MSC phenotype further comprises F4/80.

43. The method according to claim 40, wherein the phenotype includes at least one additional marker selected from the group consisting of CD31, c-kit, VEGF-receptor, and CD40.

44. The method according to claim 40, wherein the HSCs are genetically modified to overexpress Gr-1.

45. The method according to claim 40, wherein the HSCs are genetically modified to overexpress CD115.

46. The method according to claim 40, wherein the HSCs are genetically modified to overexpress F4/80.

47. The method of claim 40 wherein the HSCs are further cultured in the presence of a factor selected from the group consisting of GM-CSF, M-CSF, G-CSF, Flit-3 ligand and tumor-conditioned medium.

48. The method according to claim 40, further comprising the step of isolating the MSCs.

49. The method according to claim 48 wherein the step of isolating is by gradient centrifugation.
